(19) United States
(12) Patent Application Publication
(10) Pub. No.: US 2026/0154555 A1
(43) Pub. Date: Jun. 4, 2026

(54) MACHINE LEARNING FOR EARLY DETECTION, PREDICTION, AND PROACTIVE INTERVENTION IN MENTAL HEALTH CARE

(71) Applicant: Behzad Ranjbari Baghmisheh, Addison, TX (US)

(72) Inventor: Behzad Ranjbari Baghmisheh, Addison, TX (US)

(21) Appl. No.: 19/406,618

(22) Filed: Dec. 2, 2025

Related U.S. Application Data
(60) Provisional application No. 63/726,673, filed on Dec. 2, 2024.

(51) Int. Cl. G06N 3/0895; G16H 50/30

(52) U.S. Cl. CPC G06N 3/0895; G16H 50/30

(57) ABSTRACT

Systems and methods of analyzing and predicting emotion changes for early detection, prediction, and proactive intervention in mental health care are provided. A method for analyzing and predicting emotional dynamics may include collecting, into a sequence-modeling computing system, a plurality of information channels comprising contemporaneous Instant Emotional State Information (IESI) captured over time from an individual and further data from the individual; training, by the sequence-modeling computing system, a set of model parameters using the plurality of information channels, wherein the set of model parameters are jointly learned through a multi-task learning process applied across the plurality of information channels; and performing machine-learning analysis configured to generate at least one detection, prediction, or intervention-related output and to facilitate at least one early detection, prediction, and proactive intervention in mental-health contexts based on the IESI and the further data.

### CROSS-REFERENCE

This application claims the benefit of U.S. Provisional Application No. 63/726,673, filed Dec. 2, 2024, which application is incorporated herein by reference.

### BACKGROUND

Traditional mental health care is largely reactive—clinicians typically intervene after symptoms have worsened or crises have already occurred. In contrast, proactive intervention focuses on anticipating and addressing risks early, before acute symptoms manifest or escalate. Proactive intervention can lessen the severity of mental health challenges, improve treatment outcomes, and lower healthcare costs by reducing the need for intensive therapy sessions or emergency medical interventions.

### SUMMARY

Proactive intervention focuses on anticipating and addressing risks early, before acute symptoms manifest or escalate. By continuously analyzing real-time and historical emotional data, trained neural network model can detect emerging risks, predict future scenarios, trigger automated flagging systems, deliver timely interventions, and generate personalized care recommendations and plans. This proactive approach can empower caregivers, helping to reduce emotional crises and enhance the overall effectiveness of mental health care.

In this disclosure, system and methods intended for machine-trained analysis and predictions of emotion changes for early detection, prediction, and proactive intervention specifically tailored to mental health care are described. A computing device collects a plurality of contemporaneous Instant Emotional State Information (IESI) over time, from an individual, from one or multiple pre-existing audio-video Emotion Recognition System (ERS), wherein the pre-existing audio-video ERS can be additionally benefited from further modalities including various contemporaneous physiological data. A multilayered machine learning computing system learns trained weights using the collected series of IESI, wherein the learning can facilitate early detection, prediction, and proactive intervention in mental health care. Further contemporaneous IESI based on audio-only and video-only can be collected separately into the computing device.

The computing device learns trained weights using the further IESI, wherein the trained weights are trained through multi-task learning. The computing device analyzes the further IESI using the trained weights to perform further analysis including consistency examination, as well as to facilitate early detection, prediction, and proactive intervention in mental health care, based on the further IESI. The collected series of IESI are mapped into a Multilayer Emotion Representation Framework (MERF), before they are used for analysis using trained neural network. While it appears as of now there isn't currently a universally accepted representation of emotions however, the concept of MERF involves developing its own unique representation. Further data are collected into a second computing device, wherein the computing device learns trained weights using the further data wherein the trained weights are trained through multi-task learning. In an aspect, further data may be considered as complementary, supplemental, and/or additional data.

The computing device analyzes the further data using the trained weights to provide further information including critical cues in order to facilitate early detection, prediction, and proactive intervention in mental health care, based on the further data. Further data can include various contemporaneous physiological data. In embodiments, the further data can include one or more of contemporaneous physiological data. In some embodiments, the computing device and the second computing device are a same computing device.

In some implementations, the systems and methods may be integrated into an electronic health record database and provide clinician support. This disclosure, comprises a system and methods intended for machine-trained analysis and predictions of emotion changes for early detection, prediction, and proactive intervention in mental health care, wherein early detection, prediction, and proactive intervention can include multiple applications such as providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans.

In some implementations, a method of providing early detection, prediction, or an alert may comprise capturing two or more categories of data from an individual; collecting further data obtained from one or more sensors; collecting Instant Emotional State Information (IESI) obtained from one or multiple Emotion Recognition Systems (ERS); collecting further IESI obtained from the ERS; providing the plurality of captured data as input sequences to a machine-learning computing system; processing the input sequences using at least one trained model configured to perform at least one of early detection, prediction, or proactive-intervention analysis; and outputting the at least one early detection, prediction, or alert based on the proactive-intervention analysis to an output interface and/or a user-interaction system.

In some implementations, a method of detecting and predicting emotion changes for early detection, prediction, and proactive intervention in mental health care may comprise real-time monitoring of one or more emotional dynamics; deriving one or more insights based on the one or more emotional dynamics generating one or more predictive analytics from the monitored one or more emotional dynamics and derived one or more insights; identifying one or more target emotional indicators, trends, patterns, and risks based on the one or more detection and predictive analytics; predicting one or more target emotional indicators, trends, patterns, and risks based on the one or more predictive analytics; generating at least one alert or flag signal based on identified or predicted one or more target emotional indicators, trends, patterns, and risks; generating one or more proactive intervention recommendations; generating one or more care-related guidance or recommendations; generating one or more personalized or adaptive care plans; constructing one or more real-time analytical summaries based on the generated one or more proactive intervention recommendations, one or more care-related guidance or recommendations, or one or more personalized or adaptive care plans; displaying a visualization of analysis results of the one or more real-time analytical summaries; and exporting or transmitting the analysis results in one or more data formats.

In some implementations, a method for predicting emotional dynamics may comprise collecting Instant Emotional State Information (IESI) from individuals using audio-video Emotion Recognition Systems (ERS); analyzing said IESI using a multilayered machine learning framework with neural networks and attention mechanisms; encoding said IESI into a Multilayer Emotion Representation Framework (MERF); employing semi-supervised and multi-task learning techniques for emotion analysis; and predicting emotional indicators and trends based on the analyzed IESI.

In some implementations, a system for analyzing emotional dynamics may comprise a data collection module configured to collect Instant Emotional State Information (IESI) from individuals via pre-existing audio-video Emotion Recognition Systems (ERS), wherein said IESI includes audio-only, video-only, and physiological inputs; a multilayered machine learning framework employing neural networks with Long Short-Term Memory (LSTM) networks and attention mechanisms for analyzing said IESI; an encoding module mapping said IESI into a Multilayer Emotion Representation Framework (MERF) incorporating categorical labels, continuous valence-arousal space, and graph-based relational structures; a learning module utilizing semi-supervised and multi-task learning techniques for performing emotion analysis; and a predictive analysis module configured to predict emotional indicators and trends derived from said IESI.

In some implementations, the system may further comprise a prediction output module providing early detection alerts, predictive analytics, and real-time monitoring of emotional dynamics. In some implementations, the system may further comprise an intervention module configured to offer proactive interventions and personalized care plans based on the predictive analysis. In some implementations, the intervention module generates clinician alerts and recommends treatment adjustments.

In some implementations, a method for analyzing, identifying, and predicting emotional features related to proactive intervention may comprise collecting, into a sequence-modeling computing system, a plurality of information channels comprising contemporaneous Instant Emotional State Information (IESI) captured over time from an individual and further data from the individual; training, by the sequence-modeling computing system, a set of model parameters using the plurality of information channels, wherein the set of model parameters are jointly learned through a multi-task learning process applied across the plurality of information channels; and performing machine-learning analysis configured to generate at least one detection, prediction, or intervention-related output and to facilitate at least one early detection, prediction, and proactive intervention in mental-health contexts based on the IESI and the further data. The training may facilitate early detection. The training may facilitate prediction.

The IESI may include an emotional type or an intensity of the emotional type. The further data may include contemporaneous physiological data. The plurality of information channels may comprise further IESI.

In some implementations, a system for analyzing emotional dynamics may comprise a sequence-modeling computing system configured to receive a plurality of information channels including Instant Emotional State Information (IESI) from an individual and further data from one or more sensors associated with the individual, to perform a multi-task learning process including a set of model parameters using the plurality of information channels, wherein the set of model parameters are jointly trained across the plurality of information channels; and to perform machine-learning analysis configured to generate at least one detection, prediction, or intervention-related output and to facilitate at least one early detection, prediction, and proactive intervention in mental-health contexts based on the IESI and the further data. The detection may include detection of one or more emotional features. The prediction may include prediction of one or more emotional features.

In some implementations, a method for detecting and predicting emotional features for at least one early detection, prediction, and proactive intervention in mental-health contexts may comprise real-time monitoring of one or more emotional features; deriving one or more real-time insights based on the one or more emotional features; generating one or more predictive analytics from one or more emotional features and derived one or more insights; identifying one or more target emotional indicators, trends, patterns, and risks based on the one or more emotional features and derived one or more insights; predicting one or more target emotional indicators, trends, patterns, and risks based on the one or more predictive analytics; generating at least one alert or flag signal based on identified or predicted one or more target emotional indicators, trends, patterns, and risks; generating one or more proactive intervention recommendations; generating one or more care-related guidance or recommendations; generating one or more personalized or adaptive care plans; constructing one or more real-time analytical summaries based on one or more real-time insights the generated one or more proactive intervention recommendations, one or more care-related guidance or recommendations, or one or more personalized or adaptive care plans; displaying a visualization of analysis results of the one or more real-time analytical summaries; and exporting or transmitting the analysis results in one or more data formats.

The one or more emotional features may include at least one of a pattern, an indicator, a trend, and a risk. A pattern may include at least one of a recurring or statistically significant combination of emotional, behavioral, linguistic, physiological, or cognitive signals detected over time. A pattern is at least one of a symptom-cluster pattern, disorder-like pattern, or recurrent behavioral regularities.

An indicator may be at least one of a discrete signal, cue, or measurable attribute that reflects an emotional or mental-state characteristic at a given time. An indicator may be at least one of an emotional cue, physiological arousal, consistency or discrepancy markers, mismatch between verbal reports and expressed affect, or trigger-related emotional indications.

A trend may include a directional or progressive change in emotional or behavioral features across multiple timepoints. A trend may include at least one of a symptom drift, a gradual movement from mild to moderate anxiety-related feature, an incremental accumulation of risk markers, a progressive increase in hopelessness indicators, improvement trajectories, or a reduction in avoidance-related signals.

A risk may include at least one of a predicted probability or likelihood of an undesirable future mental-health-related outcome based on detected features, patterns, or trends. A risk may include at least one of acute risk, emergent indicators associated with crisis, self-harm propensity, relapse risk, feature constellations predictive of recurrence of a depressive or anxiety-related state, treatment-discontinuation risk, signals correlated with decreased engagement, escalation risk, or projections indicating progression toward severe impairment or functional decline.

### BRIEF DESCRIPTION OF THE DRAWINGS

FIG. 1 is an overview diagram for machine-trained analysis and predictions of emotion changes for early detection, prediction, and proactive intervention in mental health care according to some implementations.

FIG. 2A illustrates a method of identifying and predicting features related to proactive intervention according to some implementations.

FIG. 2B illustrates examples of physiological data according to some implementations.

FIG. 3 is a flow diagram for training according to some implementations.

FIG. 4 illustrates the workflow encompassing data collection, computing system and output results according to some implementations.

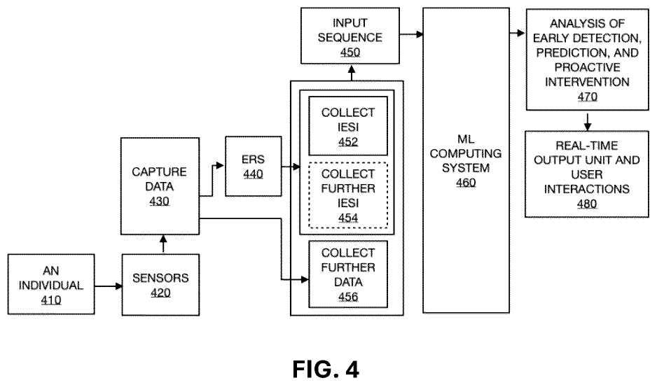

FIG. 5 illustrates the output unit and user interactions according to some implementations.

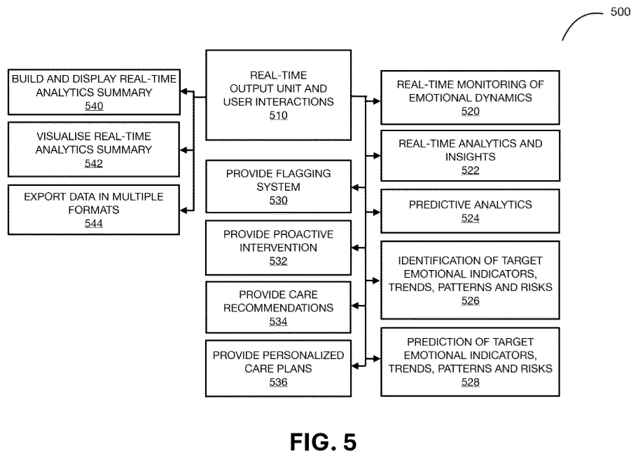

FIG. 6 illustrates the hardware components of machine learning infrastructure according to some implementations.

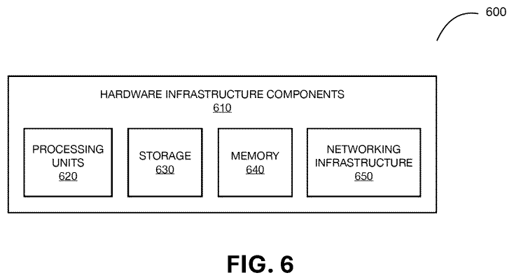

FIG. 7 illustrates a screenshot of a sample real-time output interface and user-interaction system according to some implementations.

FIG. 8 illustrates a screenshot of a sample real-time output interface and user-interaction system showing the Symptoms Analysis window according to some implementations.

FIG. 9 illustrates a screenshot of a sample real-time output interface and user-interaction system showing the Diagnostic Assessment window according to some implementations.

### DETAILED DESCRIPTION

The study of emotion dynamics emerged from the recognition that emotions are not static but rather dynamic phenomena that evolve and change over time. This field of research integrates principles from psychology, neuroscience, sociology, and other disciplines to investigate the temporal dynamics of emotions and their impact on human behavior, social interactions, and well-being. Emotion changes and human communication are interconnected aspects of social and psychological interactions. Emotion changes refer to the variations, fluctuations, transitions, and developments in human's emotional experiences over time. These changes can occur in response to internal factors (such as thoughts, memories, or physiological states) or external stimuli (such as events, interactions, or environmental cues).

Emotion changes emphasize the dynamic and temporal nature of emotions, recognizing that emotional states are not static but evolve and transition in response to evolving circumstances. Emotions can also vary in terms of intensity and duration. Some emotions may be short-lived, such as a brief feeling of anger or frustration, while others may be more prolonged, such as feelings of love or happiness.

Emotions can also be expressed in various ways, including through verbal and nonverbal communication. Verbal communication can include words, tone of voice, and other verbal cues, while nonverbal communication can include facial expressions, body language, and other physical cues. On the other hand, absolute emotions refer to discrete emotional states experienced at a particular moment in time, without consideration for their temporal context or how they may evolve over time. Absolute emotions focus on identifying and categorizing specific emotional states, such as happiness, sadness, anger, fear, or disgust, at a single point in time. In other words, studying emotion dynamics offers a more comprehensive understanding of the temporal dynamics, contextual influences, and adaptive functions of emotions, compared to a static analysis of absolute emotional states.

Emotion changes are mediated by physiological arousal, cognitive appraisals, and neural mechanisms involved in emotion processing. Physiological responses, such as changes in heart rate, respiratory patterns, skin conductance and muscle activity, can accompany shifts in emotional states. Cognitive interpretations and attributions influence the subjective experience and interpretation of emotions. The study of emotion dynamics seeks to uncover the processes, underlying mechanisms triggers, and patterns of emotional experiences over time.

There are several key aspects explored in the study of emotion dynamics. For example, temporal patterns are crucial as they can provide information on how emotions vary over time. The study of temporal patterns of emotional experiences, includes how emotions evolve over different time scales. This involves looking at short-term fluctuations, such as moment-to-moment changes in emotional states, as well as longer-term patterns, like how emotions evolve over hours, days, or even months. This includes studying patterns of emotional arousal, valence, and variability within individuals and across populations. The study of emotion dynamics can involve also the study of variations in emotional intensity (e.g., strength of emotional response) and valence (e.g., positive or negative emotion) over time. This involves tracking changes in emotional states and understanding factors that influence fluctuations in emotional intensity and valence.

The study of emotion dynamics can involve also the study of transitions and trajectories by means of focusing on transitions between different emotional states and trajectories of emotional change over time. This involves studying triggers and mechanisms of emotional transitions (e.g., stressors, coping strategies) and identifying patterns of emotional change. The study of emotion dynamics can involve also the study of contextual influences in shaping emotional experiences and expressions. This includes studying how situational cues, social interactions, cultural norms, and environmental stimuli influence the dynamics of emotions. The study of emotion dynamics can involve also the study of individual differences in emotion dynamics, exploring how factors such as personality, gender, age, and cultural background impact the way emotions unfold over time. This involves studying both within-person variability (e.g., differences in emotional responses across situations) and between-person differences (e.g., personality profiles associated with specific emotion dynamics patterns). The study of emotion dynamics using machine learning involves applying computational techniques to analyze, model, and understand the complex and dynamic nature of emotions. By leveraging computational techniques, large-scale datasets can be analyzed, temporal dependencies can be modeled, and how emotions evolve over time in various contexts can be predicted. This interdisciplinary approach holds promise for advancing both scientific understanding and a variety of practical applications.

The study of emotion dynamics is a multidisciplinary field that focuses on understanding how emotions change and unfold over time. This area seeks to uncover the processes, underlying mechanisms triggers, and patterns of emotional experiences over time. There are several key aspects explored in the study of emotion dynamics. For example, temporal patterns are crucial as they can provide information on how emotions vary over time. The study of temporal patterns of emotional experiences, can include how emotions evolve over different time scales. This involves looking at short-term fluctuations, such as moment-to-moment changes in emotional states, as well as longer-term patterns, like how emotions evolve over hours, days, or even months. This includes studying patterns of emotional arousal, valence, and variability within individuals and across populations. The study of emotion dynamics can involve also the study of variations in emotional intensity (e.g., strength of emotional response) and valence (e.g., positive or negative emotion) over time. This involves tracking changes in emotional states and understanding factors that influence fluctuations in emotional intensity and valence. The study of emotion dynamics can involve also the study of transitions and trajectories by means of focusing on transitions between different emotional states and trajectories of emotional change over time. This involves studying triggers and mechanisms of emotional transitions (e.g., stressors, coping strategies) and identifying patterns of emotional change.

The study of emotion dynamics can involve also the study of contextual influences in shaping emotional experiences and expressions. This includes studying how situational cues, social interactions, cultural norms, and environmental stimuli influence the dynamics of emotions. The study of emotion dynamics can involve also the study of individual differences in emotion dynamics, exploring how factors such as personality, gender, age, and cultural background impact the way emotions unfold over time. This involves studying both within-person variability (e.g., differences in emotional responses across situations) and between-person differences (e.g., personality profiles associated with specific emotion dynamics patterns). Importantly, the study of emotion dynamics also examines how certain maladaptive or dysregulated emotional patterns are grounded in mental disorders such as depression, anxiety, bipolar disorder, or borderline personality disorder. Understanding these atypical emotion dynamics provides insight into the mechanisms underlying psychopathology and informs the development of targeted interventions and personalized treatment approaches.

### Flow 100

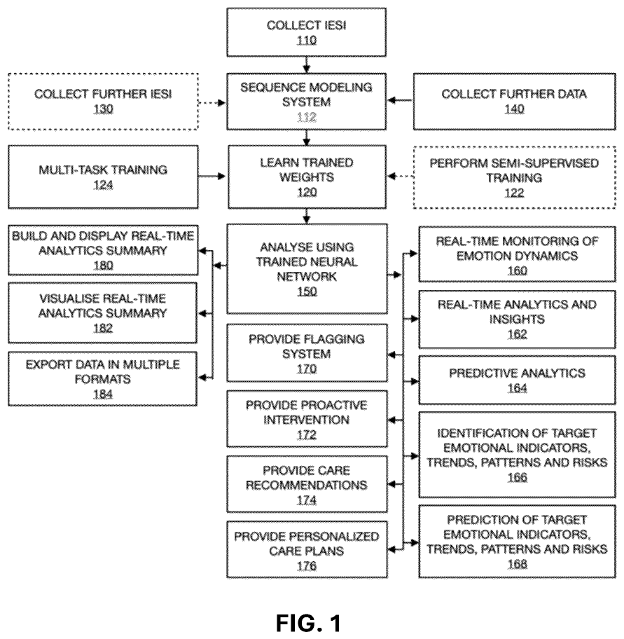

Turning to FIG. 1, Flow 100 illustrates a flow diagram for machine-trained analysis for early detection, prediction, and proactive interventions in mental health care. The flow 100 includes collecting into a computing device, a plurality of contemporaneous IESI 110 over time, from an individual, from one or multiple pre-existing audio-video Emotion Recognition Systems (ERS), wherein the pre-existing audio-video ERS can be additionally benefited from further modalities including various contemporaneous physiological data.

The computing device can vary in terms of computing power, mobility, and specific use cases and can include desktop computers, laptops, ultrabooks, All-in-One PCs, tablets, smartphones, cloud servers and virtual machines, local servers, remote servers, distributed servers, workstations, mainframes, embedded systems, wearable devices, smartwatches, IoT (Internet of Things) devices, single-board computers, edge computing devices, or any other applicable types. Emotions coming from an individual can refer to the emotional states that are experienced by an individual. These emotions can range from happiness, joy, and love, to anger, fear, and sadness as examples. Emotions can be triggered by a variety of internal and external stimuli, such as thoughts, memories, or events in the environment.

Emotions can also vary in terms of intensity and duration. Some emotions may be short-lived, such as a brief feeling of anger or frustration, while others may be more prolonged, such as feelings of love or happiness. Emotions can also be expressed in various ways, including through verbal and nonverbal communication. Verbal communication can include words, tone of voice, and other verbal cues, while nonverbal communication can include facial expressions, body language, and other physical cues. Emotions coming from an individual can be expressed through a variety of verbal and nonverbal behaviors and can provide valuable information about a person's feelings and intentions, influencing how they interact with others. Emotions can vary in intensity, from mild to strong. The same emotional state can feel different in strength to different individuals, depending on personal circumstances and context.

Although Emotions are universal across cultures and across the lifespan, the ways in which they are expressed can vary culturally. Moreover, emotions can last from a split second to several minutes or even longer, depending on the situation and individual. Emotions are complex, involving multiple physiological and psychological responses, and are interconnected, with different emotions often influencing or triggering one another. For example, anger can sometimes lead to sadness or guilt. Emotions can change quickly in response to new stimuli. They can also interact with and influence each other, making human emotions a complex and dynamic aspect of the human experience.

### Instant Emotional State Information (IESI)

IESI can include a plurality of instantaneous information of human emotional states coming from an individual. IESI can include an emotional type. IESI can include intensity of the emotional type. IESI can include the probability of occurrences of the emotional type. IESI can include more than one emotional type at the same time. IESI can be collected in real-time or near-real-time. In some specific use cases IESI can be collected even shifted in time depending on the specific task application. IESI can be collected in various time series data formats and can be represented in various formats including time-value tables, time-value arrays, time-value matrices, time-value series, or any other applicable data format. The choice of time series data format can depend on the specific application and the requirements of the analysis. IESI can be collected in various file formats including CSV (Comma Separated Values), TSV (Tab Separated Values), JSON (JavaScript Object Notation), XML (eXtensible Markup Language), HDF5 (Hierarchical Data Format 5), NPY (NumPy Array File), or any other applicable file formats suitable for representing input sequence data.

### Emotion Recognition System (ERS)

Any preferred choice of file format can depend on the specific tasks, the size and complexity of the data, the computational resources available, and the requirements for data exchange and sharing. Emotion Recognition System (ERS) can use various techniques to identify and recognize a person's emotional state. ERS can be divided into two main categories: rule-based and data-driven. Rule-based systems can use predefined rules to identify emotions based on specific cues, while data-driven systems can use machine learning techniques to learn from data and identify emotions. However, the recognition of emotions is a complex task that requires advanced machine learning techniques to extract meaningful information from the data, and the results may vary depending on the quality of the data, the choice of the physiological data and the algorithm used. The goal of most of such ERS is to produce labels that would match the labels a human perceiver would give in the same situation. IESI, can be collected from one or multiple ERS.

In an example, ERS may include one or multiple pre-existing external ERS devices. In an example, ERS may include embedded ERS system wherein the embedded ERS system can interface with a variety of sensors to interact with the physical world. In an example, ERS may include integrated ERS programming code, or any other applicable pre-existing ERS types.

A typical audio-video emotion recognition system can be based on contemporaneous audio and video information including the synchronized speech and facial expressions of individuals. However, the synchronization should not be essentially exactly simultaneous, because vocal and facial expressions can be intimately related while also being somewhat offset in time. Various types of audio-video ERS can be used, including those are additionally benefited from one or multiple contemporaneous physiological data.

The ERS deployed may provide instantaneous information of human emotional states over time. Collected output data from pre-existing ERS may be input into the computing device. In other words, a plurality of IESI is collected over time, from one or multiple pre-existing audio-video ERS, wherein the pre-existing audio-video ERS can be additionally benefited from further modalities including various contemporaneous physiological data. ERS mentioned can include one or multiple pre-existing external ERS devices. ERS mentioned can include embedded ERS system wherein the embedded ERS system can interface with a variety of sensors to interact with the physical world. ERS mentioned can also include integrated ERS programming code, or any other applicable pre-existing ERS types.

To provide a more robust estimation of the subject's emotional state, the pre-existing audio-video ERS can be benefited from a variety of data modalities including audio data and speech, video data including facial expressions, textual data such as written text and transcriptions of speech, physiological data such as heart rate variability, facial muscle activity, skin temperature, body posture, and gestures information, blood pressure, respiration rate and depth, eye movement, electroencephalogram (EEG), electrodermal activity (EDA), or any other applicable physiological data, provided by one or multiple pre-existing ERS. The flow 100 includes collecting into a computing device, a plurality of IESI over time, from one or multiple pre-existing audio-video ERS, wherein the pre-existing audio-video ERS can be additionally benefited from further modalities including various contemporaneous physiological data.

In some implementations, a method is provided including steps of collecting, into a sequence-modeling computing system, a plurality of information channels comprising: (a) contemporaneous Instant Emotional State Information (IESI) captured over time; and (b) additional data sources (“further data”); learning model parameters or weights using the contemporaneous IESI and the further data, wherein the weights are jointly learned through a multi-task learning process applied across the plurality of information channels; and providing machine-learning analysis using at least one trained neural network configured to generate detections, predictions, or intervention-related outputs that facilitate early detection, prediction, and proactive intervention in mental-health contexts based on the IESI and the further data.

In some implementations, the method may further comprise: collecting additional contemporaneous IESI (“further IESI”) into the sequence-modeling computing system; learning additional model parameters through multi-task learning applied to the further IESI; performing additional neural-network-based analysis on the further IESI; and providing additional machine-learning analysis configured to further facilitate early detection, prediction, and proactive intervention in mental-health contexts based on the further IESI. In some implementations, the method may comprise performing semi-supervised training. In an aspect, further IESI may be considered as complementary, supplemental, and/or additional IESI.

A multilayered machine learning computing system learns trained weights using the collected series of IESI over time. The flow 100 includes learning, on a sequence modeling system, trained weights 120, using the collected IESI over time, where the learning can facilitate early detection, prediction, and proactive intervention in mental health care. 120 includes learning trained weights, wherein learning trained weights can refer to the process of adjusting the parameters or weights of a model during the training phase. The process of learning trained weights is at the core of machine learning. The sequence modeling system can include neural networks, where the neural network can be part of machine learning techniques. The sequence modeling system can include attention mechanisms. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks. “Model parameters” may include any learnable numerical values within the sequence-modeling computing system and can comprise, without limitation, neural-network weight matrices, bias vectors, embedding vectors for tokens or input modalities, recurrent-state parameters such as LSTM or GRUgate coefficients, attention-projection parameters including query/key/value matrices, layer-normalization parameters, learned positional or temporal encoding parameters, feed-forward network parameters, mixture-of-experts routing parameters, and latent-state variables representing internal learned representations.

An example of sequence model computing methods leveraging attention mechanisms can involve Long Short-Term Memory (LSTM) networks with attention models. LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts.

In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data. As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights. Loss functions can be used to quantify the difference between the predicted outputs and the actual labels.

For labeled data, the loss function compares the predicted outputs with the true labels. For unlabeled data, the loss function may incorporate additional regularization terms to encourage smoothness or consistency in predictions. As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation. Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. The flow 100 includes collecting into a computing device, a plurality of IESI over time, from one or multiple pre-existing audio-video ERS, wherein the pre-existing audio-video ERS can be additionally benefited from further modalities including various contemporaneous physiological data. Sequence modeling systems can be trained using supervised, semi-supervised, or unsupervised learning approaches, depending on the availability and nature of labeled and unlabeled data.

A multilayered machine learning computing system learns trained weights using the collected series of IESI over time. Semi-supervised learning 122 is a machine learning technique that utilizes a combination of labeled and unlabeled data to train models. This approach offers several benefits compared to traditional supervised learning. In supervised learning, the model is trained on a dataset that includes input-output pairs, where the output is the correct label or value for the input. In contrast, semi-supervised learning uses a dataset that includes some input-output pairs (labeled data) and some inputs without the corresponding outputs (unlabeled data). The main idea behind semi-supervised learning is to use the unlabeled data to improve the generalization ability of the model by leveraging the additional information that it provides. The model is trained on the labeled data to learn the mapping between inputs and outputs, and then it is fine-tuned on the unlabeled data to improve its accuracy.

Semi-supervised learning can improve the performance of models by leveraging the additional information from unlabeled data. This can be particularly useful in cases where labeled data is scarce or expensive to obtain. By using unlabeled data, semi-supervised learning can reduce the amount of annotation required to train a model. This can be more cost-effective and efficient than relying solely on labeled data. Semi-supervised learning can be useful for handling complex and high-dimensional data. It can be also used to handle imbalanced datasets, where the proportion of samples in different classes is unbalanced. Semi-supervised learning can be used to handle datasets with noisy labels or missing data, by using the unlabeled data to help the model distinguish between good and bad labels. However, the performance of semi-supervised learning is highly dependent on the quality and quantity of the unlabeled data.

Multi-task training 124 is a technique used in sequence modeling that allows a model to learn multiple tasks at the same time. This approach is also known as simultaneously learning. The idea behind multi-task learning is that a model trained on multiple tasks can learn shared representations or features that can be useful for all the tasks, and this can improve the performance of the model on each task. This can result in models that capture richer representations of the input sequence and achieve better performance on unseen data. It can also lead to faster convergence during the optimization process. Among various approaches multi-task learning can include multi-task learning with attention wherein attention mechanisms can be used to selectively focus on different parts of the input sequence for different tasks wherein this approach allows the model to learn different representations for each task.

The flow 100 includes collecting into a computing device, a plurality of IESI over time, from one or multiple pre-existing audio-video ERS, wherein the pre-existing audio-video ERS can be additionally benefited from further modalities including various contemporaneous physiological data. A multilayered machine learning computing system learns trained weights using the collected series of IESI over time. The flow 100 includes collecting further IESI 130 into the computing device.

The further IESI can include contemporaneous IESI based on audio-only and contemporaneous IESI based on video-only which each can be collected separately into the computing device in which, the computing device learns trained weights using the further IESI, wherein the trained weights are trained through multi-task learning.

The computing device can analyze the further IESI using trained weights to perform further analysis including consistency examination 132, as well as to facilitate early detection, prediction, and proactive intervention in mental health care, based on the further IESI. Consistency examination can be a crucial step in the process of analysis of emotion dynamics as it can help to ensure that the data being collected and used are reliable and valid, as any inconsistent data can lead to inaccurate conclusions. Consistency examination can also help to identify any potential sources of bias or error in the data collection and analysis process. Consistency examination can further help to identify mental health indicators, trends and patterns such as symptoms and disorders in the data that may not be immediately apparent. Consistency examination can involve comparing data collected from different sources, such as data collected from contemporaneous IESI based on audio-only and contemporaneous IESI based on video-only, to ensure that they are consistent with each other and provide a clear and consistent picture of the emotional state. IESI based on audio-only can represent verbal communication, wherein IESI based on video-only can represent nonverbal communication.

Verbal communication can include words, tone of voice, and other verbal cues, while nonverbal communication can include facial expressions, body language, and other physical cues. A computing device collects a plurality of contemporaneous IESI over time, from an individual, from one or multiple pre-existing audio-video ERS, wherein the pre-existing audio-video ERS can be additionally benefited from further modalities including various contemporaneous physiological data.

FIG. 1 illustrates an overview diagram for analysis and predictions of emotion changes related to early detection, prediction, and proactive intervention in mental health care. In some implementations, the method may include steps of collecting plurality of contemporaneous IESI 110 and “further data” 140 into a sequence modeling computing system 112; learning weights 120 through multi-task learning 124 based on plurality of contemporaneous IESI and “further data”; performing analysis using trained neural network 150; facilitating early detection, prediction, and proactive intervention based on IESI and further data.

In some implementations, the method may include collecting further contemporaneous IESI (“further IESI”) 130, into a sequence modeling computing system 112; learning weights 120 through multi-task learning 124 based on further IESI; performing further analysis using trained neural network 150; and facilitating early detection, prediction, and proactive intervention based on further IESI. In some implementations, the method may include performing semi-supervised training 122.

A multilayered machine learning computing system learns trained weights using the collected series of IESI over time. The flow 100 includes collecting further IESI into the computing device. The further IESI can include contemporaneous IESI based on audio-only and contemporaneous IESI based on video-only which each can be collected separately into the computing device in which, the computing device learns trained weights using the further IESI, wherein the trained weights are trained through multi-task learning. The computing device can analyze the further IESI using trained weights to perform further analysis including consistency examination, as well as to facilitate early detection, prediction, and proactive intervention in mental health care, based on the further IESI. The flow 100 includes capturing further data 140, into a second computing device wherein the computing device can learn trained weights using the further data wherein the trained weights are trained through multi-task learning. The second computing device can analyze the further data using trained weights to provide further information including for example emotional cues 142 in order to facilitate early detection, prediction, and proactive intervention in mental health care based on the further data.

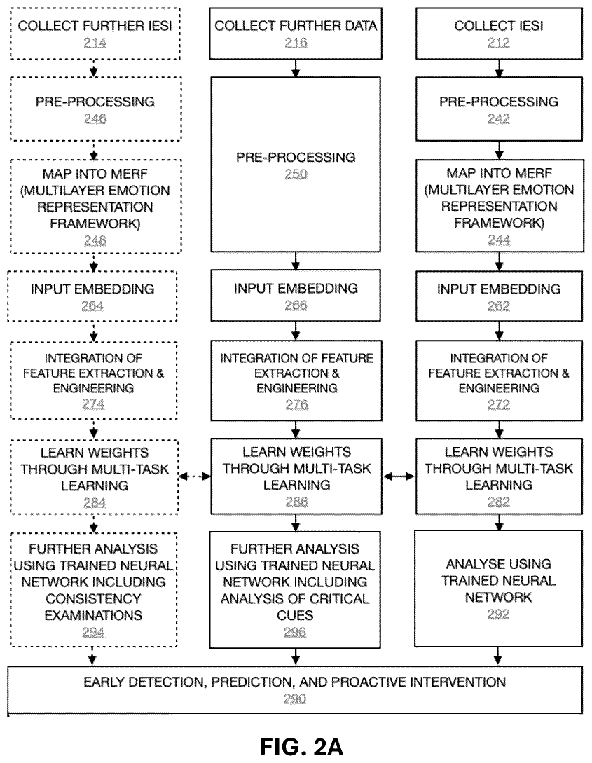

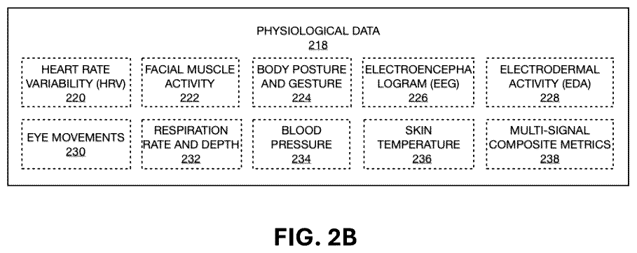

Further data can include various contemporaneous physiological data, such as contemporaneous heart rate variability data, facial muscle activity, skin temperature, blood pressure, respiration rate and depth, eye movement, electroencephalogram (EEG), electrodermal activity (EDA), body posture, and gestures information, multi-signal composite metrics, or any other applicable physiological data (FIG. 2B). In embodiments, further data can include one or more contemporaneous physiological data. The second computing device can vary in terms of computing power, mobility, and specific use cases and can include a desktop computer, a laptop, an ultrabook, All-in-One PC, a tablet, a smartphone, a cloud server and virtual machine, a local server, a remote server, a distributed server, a workstation, a mainframe, an embedded system, a wearable device, a smartwatch, an IoT (Internet of Things) device, a single-board computer, an edge computing device, or any other applicable types. In embodiments, the computing device and the second computing device are a same computing device.

In an aspect, performing analysis using trained neural network 150 can include multilayered machine-trained sequence modeling system, where the sequence modeling system can include neural networks, where the neural network can be part of machine learning techniques. The sequence modeling system can include attention mechanisms.

In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks.

An example of sequence model computing methods leveraging attention mechanisms can involve Long Short-Term Memory (LSTM) networks with attention models. LSTM networks, are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts.

In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data.

As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights. Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions.

As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation. Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data.

Analysis using trained neural network can include providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans, etc. In the example of LSTM networks with attention models the model can maintain an internal state that evolves over time, allowing it to capture dependencies and patterns across different time steps. As the sequence model processes the input data, it can learn to identify patterns and regularities that are characteristic of the underlying data distribution. After training the sequence model, the learned patterns can be evaluated and interpreted to assess the model's performance and understand the underlying data. The flow 100 includes collecting into a computing device, a plurality of IESI over time, from one or multiple pre-existing audio-video ERS, wherein the pre-existing audio-video ERS can be additionally benefited from further modalities including various contemporaneous physiological data.

A multilayered machine learning computing system learns trained weights using the collected series of IESI over time, wherein the learning can facilitate early detection, prediction, and proactive intervention in mental health care. Further contemporaneous IESI based on audio-only and video-only can be collected separately into the computing device. The computing device can learn trained weights using the further IESI, wherein the trained weights are trained through multi-task learning. The computing device can analyze the further IESI using trained weights to perform further analysis including consistency examination as well as to facilitate early detection, prediction, and proactive intervention in mental health care based on the further IESI.

A second computing device can collect further data wherein the computing device learns trained weights using the further data wherein the trained weights are trained through multi-task learning. The computing device can analyze the further data using trained weights to provide further information including emotional cues to facilitate early detection, prediction, and proactive intervention in mental health care based on the further data. In embodiments, the computing device and the second computing device are a same computing device.

Analysis using trained neural network can include real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans, etc. Providing real-time monitoring of emotion dynamics 160, can include real-time tracking of how emotions change over time—their patterns, speed, variability, and transitions, etc. (e.g., a steady rise in sadness, rapid emotional swings, or prolonged lack of positive affect). Absolute emotions focus on identifying and categorizing specific emotional states, such as happiness, sadness, anger, fear, or disgust, at a single point in time.

While absolute emotions refer to discrete emotional states experienced at a particular moment in time, without consideration for their temporal context or how they may evolve over time, however, studying emotion dynamics offers a more comprehensive understanding of the temporal dynamics, contextual influences, and adaptive functions of emotions, compared to a static analysis of absolute emotional states. Emotion dynamics are mediated by physiological arousal, cognitive appraisals, and neural mechanisms involved in emotion processing. Physiological responses, such as changes in heart rate, respiratory patterns, skin conductance and muscle activity, can accompany shifts in emotional states. Monitoring of emotion dynamics seeks to uncover the processes, underlying mechanisms triggers, and patterns of emotional experiences over time.

Disclosed herein include methods of identifying and predicting one or more features related to proactive intervention. In an aspect, a feature may be at least one of a pattern, an indicator, a trend, and a risk.

A “pattern” refers to a recurring or statistically significant combination of emotional, behavioral, linguistic, physiological, or cognitive signals detected over time. Non-limiting examples include Symptom-cluster patterns (e.g., co-occurrence of low affect, anhedonia, and disturbed sleep); Disorder-like patterns (e.g., feature constellations resembling depressive, anxiety-related, or trauma-related profiles); Recurrent behavioral regularities (e.g., repeated avoidance sequences or rumination loops); and Persistent linguistic-emotional patterns (e.g., stable negative self-referential language).

An “indicator” refers to a discrete signal, cue, or measurable attribute that reflects an emotional or mental-state characteristic at a given time. Non-limiting examples include Emotional cues (e.g., sudden tension, irritability, tearfulness, or physiological arousal); Consistency or discrepancy markers (e.g., mismatch between verbal reports and expressed affect); Trigger-related emotional indications (e.g., detectable shifts following specific topics, prompts, or events); Micro-signal variations (e.g., changes in tone, speech rate, or affective intensity).

A “trend” refers to a directional or progressive change in emotional or behavioral features across multiple timepoints. Non-limiting examples include Symptom drift (e.g., gradual movement from mild to moderate anxiety-related features); Incremental accumulation of risk markers (e.g., progressive increase in hopelessness indicators); Improvement trajectories (e.g., reduction in avoidance-related signals); Behavioral stabilization trends (e.g., increasing regularity in routines or emotional regulation patterns).

A “risk” refers to a predicted probability or likelihood of an undesirable future mental-health-related outcome based on detected features, patterns, or trends. Non-limiting examples include: Acute risk (e.g., emergent indicators associated with crisis, self-harm propensity, or rapid deterioration); Relapse risk (e.g., feature constellations predictive of recurrence of a depressive or anxiety-related state); Treatment-discontinuation risk (e.g., signals correlated with decreased engagement or dropout likelihood); Escalation risk (e.g., projections indicating progression toward severe impairment or functional decline).

In some implementations, a method for identifying and predicting features related to proactive intervention in mental-health care, the method may comprise collecting, from an individual, a plurality of information channels comprising: (a) contemporaneous Instant Emotional State Information (IESI) captured over time; and (b) further data comprising physiological, behavioral, linguistic, contextual, or historical information; pre-processing the IESI to clean, normalize, transform, and prepare the IESI for machine-learning analysis; pre-processing the further data to clean, normalize, transform, and prepare the further data for machine-learning analysis; mapping the pre-processed IESI into a Multilayer Emotion Representation Framework (MERF); performing input embedding on the pre-processed and mapped IESI; performing input embedding on the pre-processed further data; extracting features and performing feature-engineering operations on the embedded IESI; extracting features and performing feature-engineering operations on the embedded further data; learning model parameters through multi-task learning applied to the IESI; learning model parameters through multi-task learning applied to the further data; providing machine-learning analysis using at least one trained neural network configured to identify and predict patterns, indicators, trends, and risks using the combined IESI and further data; performing additional neural-network-based analysis to detect critical cues derived from the further data; and outputting predictions, detections, or alerts that facilitate early detection, prediction, and proactive intervention in mental-health contexts.

The method may further comprise: collecting additional contemporaneous IESI (“further IESI”); pre-processing the further IESI; mapping the further IESI into the Multilayer Emotion Representation Framework; performing input embedding on the further IESI; performing feature extraction and feature-engineering operations on the further IESI; learning additional model parameters through multi-task learning applied to the further IESI; performing additional neural-network-based analysis on the further IESI, including consistency examinations; and generating outputs that further facilitate early detection, prediction, and proactive intervention.

The method may further comprise collecting further data, wherein the further data can include contemporaneous physiological information. Collecting contemporaneous physiological information can include collecting at least one of heart rate variability (HRV), facial muscle activity, body posture and gesture information, electroencephalogram (EEG), electrodermal activity (EDA), eye movements, respiration rate and depth, blood pressure, skin temperature, multi-signal composite metrics, or any other physiological signal capable of conveying emotional, behavioral, or mental-state information.

In some implementations, a method of identifying and predicting features related to proactive intervention may include steps comprising: collecting a plurality of contemporaneous IESI 212 and “further data 216”; pre-process IESI 242 to clean and prepare the input data for analysis, making it easier for models to extract meaningful insights and patterns; pre-processing further data 250; mapping the IESI into a MERF (Multilayer Emotion Representation Framework) 244; performing input embedding on IESI 262, performing input embedding on further data 266; performing integration of feature extraction and feature engineering on IESI 272; performing integration of feature extraction and feature engineering on further data 276; learning weights through multi-task learning based on IESI 282; learning weights through multi-task learning based on further data 286; performing analysis using trained neural network 292; further analysis using trained neural network including analysis of critical cues based on further data 296, and facilitating early detection, prediction, and proactive intervention 290.

In some implementations, the method may further comprise early detection of emotional patterns, indicators, trends and risks in mental health care. In some implementations, the method may use analysis and predictions of emotional features.

In some implementations, the method may further comprise collecting further contemporaneous IESI (“further IESI”) 214; pre-processing further IESI 246; mapping the further IESI into MERF (Multilayer Emotion Representation Framework) 248; performing input embedding process on further IESI 264; performing integration of feature extraction and feature engineering on further IESI 274; learning weights through multi-task learning based on further IESI 284; performing further analysis using trained neural network including consistency examinations based on further IESI 294; and facilitating early detection, prediction, and proactive intervention 290.

In some implementations, the method may comprise collecting further data, wherein collecting further data can include collecting various contemporaneous physiological data 218. Collecting contemporaneous physiological data can include collecting one or more of contemporaneous physiological data such as heart rate variability (HRV) 220, facial muscle activity 222, body posture and gesture information 224, electroencephalogram (EEG) 226, electrodermal activity (EDA) 228, eye movements 230, respiration rate and depth 232, blood pressure 234, skin temperature 236, multi-signal composite metrics 238, or any other applicable physiological data.

This process can include pre-processing of the input data, mapping into MERF (Multilayer Emotion Representation Framework), undergoing embedding processes, followed by feature extraction, where the trained neural network converts the raw emotion signals into descriptive features used for continuous multimodal emotion dynamics tracking with physiological correlates. Providing real-time analytics and insights 162 is the foundation for delivering instant insights and responses to caregivers. In the mental health care domain, leveraging machine learning and trained neural network for real-time analytics involves the seamless processing and analysis of continuous emotional and behavioral data.

Real-time analytics can provide caregivers an immediate emotional insight to gain real-time awareness of emotional changes, fostering better decision-making and timely interventions. Moreover, caregivers receive an ongoing analysis of patient's mental health indicators, trends, patterns, and emerging risks enabling proactive mental well-being management. Providing real-time analytics using trained neural network can enable the mental health platform to effectively respond to the complexities of human emotions by acting as the foundation for both predictive and proactive mental health care. Analysis using trained neural network can include real-time monitoring of emotional dynamics, real-time analytics and insights, predictive analytics, identification of target emotional indicators, trends, patterns, and risks, prediction of target emotional indicators, trends, patterns, and risks, flagging system, proactive intervention, care recommendations, personalized care plans, etc. In the mental health care domain providing predictive analytics 164 is the foundation for proactive care and proactive intervention. Leveraging machine learning and trained neural network, predictive analytics involves forecasting future emotional indicators, trends, patterns, and identify potential risks, and proactively address emerging issues in mental health care.

Predictive analytics can provide proactive mental health management. By identifying risks early, predictive analytics can provide caregivers take preventive measures to avoid emotional crises. Predictive analytics can provide precision in emotional interventions. Predictive analytics can ensure that interventions are timely and highly relevant, improving their effectiveness. Predictive analytics can also provide reduction in healthcare costs. Early detection and intervention can reduce the need for costly treatments and hospitalizations associated with advanced mental health conditions. Predictive insights can empower caregivers with actionable knowledge, helping them stay ahead of potential mental challenges. Predictive analytics, driven by machine learning and trained neural networks, serves as a cornerstone of an AI-powered mental health care system.

By forecasting emotional indicators, trends, patterns, and identifying risks, it can enable proactive interventions, tailored support, and better mental health management. This capability can transform mental care into a forward-looking, data-driven practice, creating lasting impacts for individuals and the broader healthcare system.

Analysis using trained neural network can include providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans, etc. Analysis using trained neural network can include identification of target emotional indicators, trends, patterns, and risks 166. Target emotional indicators, trends, patterns, and risks can encompass a wide range of clinically and psychologically meaningful outcomes that are valuable for detection and monitoring in mental health care.

These outcomes can closely relate to the presence or progression of symptoms and disorders within mental health contexts. Examples of symptom-related indicators, trends, patterns, and risks can include the early detection or identification of symptoms such as depressed mood, generalized anxiety, panic symptoms, and similar manifestations. Each detected symptom can be accompanied by an associated confidence score, severity level, and information about whether it is stable, improving, or worsening over time. The model may also generate respective hourly, daily, weekly, or monthly symptom trajectories to illustrate these changes dynamically.

Examples of disorder-related indicators, trends, patterns, and risks can include the early detection or identification of mental health disorders such as Major Depressive Disorder (single or recurrent episode, moderate severity), Generalized Anxiety Disorder, or Persistent Depressive Disorder (Dysthymia). These detections can include associated confidence scores, severity levels, and temporal dynamics derived from ongoing analysis. Each of the above indicators, trends, patterns, and risks is mapped to its corresponding extracted feature within the machine learning framework. These features are processed and interpreted by trained neural network model, enabling accurate, data-driven insights into patients'emotional indicators, trends, patterns, and risks.

The mechanism of identification of target emotional indicators, trends, patterns, and risks in the context of sequence modeling can involve detecting regularities, structures, or recurring motifs within input sequence data. Before identifying indicators, trends, patterns, and risks, the raw sequence data often needs to be preprocessed and transformed into a suitable format in which the process may involve extracting features. Sequences of emotions are typically tokenized and converted into numerical representations using techniques like input embeddings. The preprocessed sequences can then be fed into a sequence model for analysis. The sequence model then can learn to capture the underlying patterns in the data by iteratively processing the input sequences.

Analyze using trained neural network can include multilayered sequence modeling system, wherein the sequence modeling system can include neural networks, where the neural network can be part of machine learning techniques. The sequence modeling system can include attention mechanisms. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks.

An example of sequence model computing methods leveraging attention mechanisms can involve Long Short-Term Memory (LSTM) networks with attention models. LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts.

In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data.

As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights. Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions.

As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation.

Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. In the example of LSTM networks with attention models the model can maintain an internal state that evolves over time, allowing it to capture dependencies and patterns across different time steps.

As the sequence model processes the input data, it can learn to identify patterns and regularities that are characteristic of the underlying data distribution. After training the sequence model, the learned patterns can be evaluated and interpreted to assess the model's performance and understand the underlying data. Analysis using trained neural network can include providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans. Analysis using trained neural network can include prediction of target emotional indicators, trends, patterns, and risks 168.

Prediction of target emotional indicators, trends, patterns, and risks can involve the application of machine learning techniques designed for Next Emotion Prediction (NEP). Using trained neural network model, the system analyzes current and historical emotional data to anticipate an individual's future emotional data. This predictive capability enables the trained neural network model, to forecast potential emotional indicators, trends, patterns, and emerging risks before they manifest. The Next Emotion Prediction process can generate one or multiple possible future scenarios, each associated with a probability or confidence level, allowing for more informed and proactive mental health interventions.

Target emotional indicators, trends, patterns, and risks can encompass a wide range of clinically and psychologically meaningful outcomes that are valuable for predictive analysis in mental health care. These outcomes can relate to the onset, progression, or recurrence of symptoms and disorders within mental health contexts. Examples of symptom-related indicators, trends, patterns, and risks can include the prediction of symptoms such as depressed mood, generalized anxiety, panic symptoms, and similar manifestations. Each predicted symptom is accompanied by an associated confidence score and severity risk level, providing interpretable, data-driven estimates of likelihood and intensity. Examples of disorder-related indicators, trends, patterns, and risks can include the prediction of mental health disorders such as posttraumatic stress disorder (PTSD), Schizophrenia, Bipolar I Disorder (Current or Most Recent Episode, Manic), obsessive-compulsive disorder (OCD), and similar manifestations.

These predictions can include associated confidence scores and severity risk assessments that quantify the model's certainty and the potential clinical impact. Each of the indicators, trends, patterns, and risks is mapped to its corresponding extracted feature within the machine learning framework. These features are then processed and interpreted by trained neural network model, enabling precise, data-driven insights into patients'emotional indicators, trends, patterns, and risks. The flow 100 includes collecting into a computing device, a plurality of IESI over time, from one or multiple pre-existing audio-video ERS, wherein the pre-existing audio-video ERS can be additionally benefited from further modalities including various contemporaneous physiological data.

A multilayered machine learning computing system learns trained weights using the collected series of IESI over time, wherein the learning can facilitate early detection, prediction, and proactive intervention in mental health care. Further contemporaneous IESI based on audio-only and video-only can be collected separately into the computing device. The computing device learns trained weights using the further IESI, wherein the trained weights are trained through multi-task learning. The computing device can analyze the further IESI using trained weights to perform further analysis including consistency examination, as well as to facilitate early detection, prediction, and proactive intervention in mental health care based on the further IESI.

A second computing device collects further data wherein the computing device learns trained weights using the further data wherein the trained weights are trained through multi-task learning. The second computing device can analyze the further data using trained weights to provide further information including critical cues to facilitate early detection, prediction, and proactive intervention in mental health care based on the further data. In embodiments, the computing device and the second computing device are a same computing device. The mechanism of next emotions prediction in the context of sequence modeling can involve predicting the next emotion or token in a sequence of emotions or tokens given the preceding context. In other words, it involves prediction of the most likely emotions that follow a given sequence of emotions, given the context provided by those emotions. This task can be addressed using various sequence modeling techniques, including LSTMs with attention models. The input to the model can consist of a sequence of emotions or tokens, represented as numerical vectors. Each emotion or token can be typically converted into a high-dimensional vector using techniques like input embeddings.

The input sequence can then feed into the model, and the model can process each emotion or token one at a time. Analyze using trained neural network can include multilayered sequence modeling system, wherein the sequence modeling system can include neural networks, where the neural network can be part of machine learning techniques. The sequence modeling system can include attention mechanisms. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks.

An example of sequence model computing methods leveraging attention mechanisms can involve Long Short-Term Memory (LSTM) networks with attention models. LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts.

In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data.

As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights.

Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions. As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation.

Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. Prediction of next emotions can enhance our comprehension of how emotions evolve over time. This approach can unveil the underlying processes, triggers, indicators, trends and patterns of emotional experiences, serving as a valuable tool for improvement. In certain scenarios, the disclosed system can aid in identifying a broad spectrum of risky situations, preemptively alerting to these risks, and assisting in their prevention.

Analysis and predictions using trained neural network can provide flagging systems 170. The mechanism of a flagging system can be used to highlight or flag specific indicators, trends, patterns or any potential risks or opportunities that are of interest or concern. The purpose of the flagging system can be to draw attention to those indicators, trends, patterns or potential risks or opportunities so that appropriate actions can be taken in advance. The flagging system for predictive analytics using sequence modeling system can analyze data in real-time and raise a flag or alert as soon as a potential issues or opportunities are predicted. Flagging rules can be defined to determine when to raise a flag based on the identified emotional indicators, trends, patterns or potential risks or opportunities.

These rules may specify conditions under which certain indicators, trends, patterns or potential risks or opportunities should be flagged, such as exceeding predefined thresholds, or indicating potential risks or opportunities. A flagging mechanism is implemented to automatically raise flags when the predefined conditions specified by the flagging rules are met. Flags can be generated in real-time as new data becomes available. Flagged indicators, trends, patterns or potential risks or opportunities can be then visualized and reported accordingly to take proactive measures to address them before they happen and become a problem. Analyze using trained neural network can include multilayered sequence modeling system, wherein the sequence modeling system can include neural networks, where the neural network can be part of machine learning techniques. The sequence modeling system can include attention mechanisms.

Analysis and predictions using trained neural network can include providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans, etc. In certain scenarios, the disclosed system can aid in identifying a broad spectrum of risky situations, preemptively alerting to these risks, and assisting in their prevention. By identifying target emotional indicators, trends and patterns and preemptively flagging critical moments, this system provides a valuable tool for early intervention, ensuring timely alerts and personalized support. Analysis and predictions using trained neural network can provide flagging system and proactive intervention 172.

Traditional mental health care is largely reactive—clinicians typically intervene after symptoms have worsened or crises have already occurred. In contrast, proactive intervention focuses on anticipating and addressing risks early, before acute symptoms manifest or escalate. By continuously analyzing real-time and historical emotional data, trained neural network model can detect emerging risks, predict future scenarios, trigger automated flagging systems, deliver timely interventions, and generate personalized care recommendations and plans. This proactive approach can empower caregivers, helping to reduce emotional crises and enhance the overall effectiveness of mental health care. Proactive intervention can lessen the severity of mental health challenges, improve treatment outcomes, and lower healthcare costs by reducing the need for intensive therapy sessions or emergency medical interventions. Proactive intervention also enables tailored, user-centric care, as recommendations and actions are personalized to each individual's unique emotional patterns, preferences, and behavioral history.

In practice, proactive intervention can anticipate symptom escalation before it occurs. Proactive intervention can prioritize high-risk patients within clinical caseloads. Proactive intervention can dynamically tailor interventions, such as adjusting medication, increasing therapy frequency, or recommending targeted coping strategies. When the system detects a rising risk, proactive intervention can take various forms depending on the severity and context, such as: providing clinician alerts (enabling early outreach before relapse), providing AI-generated emotional regulation support (offering immediate self-guided interventions), providing treatment adjustment recommendations (assisting clinicians in fine-tuning care dynamically), providing crisis prevention protocols (preventing escalation to emergency situations). By addressing subtle emotional fluctuations early, proactive interventions can stabilize emotional trajectories, reduce the likelihood of acute episodes, hospitalizations, or dropouts, and enhance long-term patient well-being.

Each proactive intervention and its outcome (e.g., improvement or no change) provides new labeled data back to the model. This creates a continuous learning loop: Detection→Prediction→Intervention→Outcome→Learning→Improved Model Accuracy. Over time, the trained neural network can learn which interventions are most effective for which profiles, leading to highly personalized and context-aware care recommendations.

This supports precision mental health care, where interventions are dynamically adapted to each individual's unique emotional and physiological indicators, trends, patterns, and risks. Providing care recommendations 174 can refer to generating data-driven, personalized, and context-aware suggestions that guide clinicians in optimizing mental health care. These recommendations are not generic “tips,” but intelligent, adaptive outputs produced by the trained neural network. They are grounded in continuous emotional monitoring, predictive analytics, and contextual interpretation of multimodal data. In other words, care recommendations can translate analytic intelligence into actionable clinical and behavioral guidance.

Care recommendations can be useful because they can bridge the gap between data insight and clinical action. Care recommendations can support mental health care by enhancing clinical decision-making by providing clinicians with AI-assisted insights on care optimizations, what to adjust or prioritize in treatment. Care recommendations can support mental health care by helping clinicians with standardize decision support when clinician availability is limited. Care recommendations can support mental health care by offering personalized care plans.

Care recommendations can support mental health care by identifying warning patterns early and suggest preventive interventions before emotional crises escalate. Care recommendations can support mental health care by encouraging adaptive coping behaviors that stabilize emotional trajectories. Care recommendations can support mental health care by helping extend clinician reach by providing automated, evidence-informed recommendations to large patient populations. Care recommendations can support mental health care by supporting scalable, data-driven care. The sequence modeling system can include neural networks, where the neural network can be part of machine learning techniques. The sequence modeling system can include attention mechanisms. Analysis and predictions using trained neural network can provide support to create personalized care plans 176.

Personalized care plans include ML-generated personalized care plans wherein, ML-generated personalized care plans are personalized care plans generated by the trained neural network. ML-generated personalized care plans can include pre-authorization ML-generated personalized care plans wherein pre-authorization ML-generated personalized care plans can include ML-generated, personalized, evidence-informed, and patient-specific treatment roadmap that can be automatically created before a clinician formally authorizes or modifies it. Personalized care plans can include multidisciplinary or integrated care. Care plans can include clinical formulation, including conceptual/diagnostic reasoning. Care plans can include treatment plans. Care plans can include psychiatric management plans including medication and symptom management. ML-generated personalized care plans can integrate real-time emotional analytics, longitudinal behavioral data, and predictive modeling results to recommend initial or adaptive care strategies, while the clinicians can remain the ultimate decision-maker. Traditional care planning in mental health usually relies on periodic assessments and manual interpretation of subjective data. This can delay interventions and reduce personalization.

By contrast, the ML-generated personalized care plan, can provide immediate plan generation based on live emotional and physiological insights. The ML-generated personalized care plan can provide clinically interpretable recommendations for treatment. The ML-generated personalized care plan can provide continuous plan adaptation as new data arrives (e.g., emotional dynamics, symptom changes, compliance signals). The ML-generated personalized care plan can provide pre-authorization workflows—clinicians can review, approve, or edit ML-suggested plans before activation. This can bridge data analytics with clinical decision support, ensuring care plans evolve with patient needs while preserving medical oversight. The benefits of ML-generated personalized care plans are multifaceted. ML-generated personalized care plans can enable immediate care plan generation as soon as a risk is detected, ensuring that early intervention can occur without delay. ML-generated personalized care plans also can provide tailored interventions that align with each patient's unique emotional, physiological, and behavioral profile, enhancing personalization and treatment relevance. Moreover, ML-generated personalized care plans can reduce the manual planning workload for clinicians by automating much of the data analysis and preliminary care design process, allowing practitioners to focus on clinical judgment and patient engagement. As emotional and behavioral data evolve over time, the system can dynamically update ML-generated personalized care plan, ensuring that recommendations remain responsive to the patient's changing condition.

Additionally, ML-generated personalized care plans can enable proactive and scalable planning across large populations, supporting healthcare systems in delivering consistent, data-informed, and adaptive mental health care at scale. Build and display real-time analytics summary 180 alongside visualizing real-time analytics summary 182 can involve presenting data insights in a clear, intuitive, and actionable manner. Effectively visualizing these data can include, for instance, the development of interactive dashboards capable of engaging users through features such as a detection module, prediction module, pre-auth care plan module, monitoring module, flagging system module, and urgent intervention module, in real-time.

These dashboards can utilize charts, graphs, and gauges to visualize data. Additionally, integrating filters and drill-down capabilities can enable users to explore data across various levels of granularity. Exporting data in multiple formats 184 can involve converting the output data into various file types or structures to accommodate different use cases and applications. Some common file types can include for example CSV (Comma-Separated Values) format, where each row represents a data point or observation, and columns represent different attributes or features. CSV files are widely supported by spreadsheet software and can be easily imported into data analysis tools for further processing. Another file type example can include JSON (JavaScript Object Notation) format, representing structured data as key-value pairs or nested objects. JSON files are commonly used for exchanging data between web services and applications and are easily readable by both humans and machines.

Another file type example can include XML (eXtensible Markup Language) documents, using tags to denote hierarchical relationships between elements. XML is suitable for representing complex data structures and is commonly used for data interchange in web services and document formats. Another file type example can include Excel Spreadsheets (.xlsx) containing the results, with each sheet representing a different subset of data or analysis. Excel files provide a familiar interface for viewing and analyzing tabular data and support various formatting options and formulas. Another file type example can include Text Files or plain text files, with each line representing a data point or record. Text files are lightweight and easily readable, making them suitable for exporting large datasets or logs. Another file type example can include Relational database management system (RDBMS) such as MySQL, PostgreSQL, or SQLite. Exporting results to a database allows for efficient storage, retrieval, and querying of structured data, supporting complex data relationships and transactions. Another file type example can include Image Files for visual representation of patterns or outputs, such as graphs, charts, or heatmaps. Image files are suitable for exporting visualizations generated by the sequence model, enabling easy sharing and integration into reports or presentations. Another file type example can include custom file formats or data structures tailored to specific requirements or downstream applications.

Custom formats can accommodate specialized data needs and may include metadata, annotations, or additional context relevant to the results. By exporting data results in multiple formats, a sequence modeling system ensures compatibility with diverse data analysis tools, visualization platforms, and downstream applications, enabling seamless integration and utilization of the model outputs across various domains and workflows.

The flow 200 involves collecting into computing devices various types of input data 210 including a plurality of IESI 212 over time, from one or multiple pre-existing audio-video ERS, wherein the pre-existing audio-video ERS can be additionally benefited from further modalities including various contemporaneous physiological data. A multilayered machine learning computing system learns trained weights using the collected series of IESI over time, wherein the learning can facilitate early detection, prediction, and proactive intervention in mental health care. Further contemporaneous IESI 214 based on audio-only and video-only can be collected separately into the computing device. The computing device learns trained weights using the further IESI, wherein the trained weights are trained through multi-task learning. The computing device can analyze the further IESI using trained weights to perform further analysis including consistency examination as well as to facilitate early detection, prediction, and proactive intervention in mental health care based on the further IESI.

A second computing device collects further data and emotional cues 216 wherein the computing device learns trained weights using the further data wherein the trained weights are trained through multi-task learning. The computing device can analyze the further data using trained weights to provide further information including for example critical cues to facilitate early detection, prediction, and proactive intervention in mental health care based on the further data. Further data can include various contemporaneous physiological data 218. In embodiments, the computing device and the second computing device are a same computing device. Physiological data can include heart rate variability (HRV), facial muscle activity, body posture and gesture information, electroencephalogram (EEG) (EEG measures electrical activity in the brain, which can be used to infer emotional states such as calm, focus, and relaxation), electrodermal activity (EDA), eye movements (can be used to infer emotions, such as the rate of blinking, gaze direction, and pupillary dilation), respiration rate and depth, blood pressure, skin temperature, multi-signal composite metrics, or any other applicable physiological data.

In embodiments, the further data can include one or more of contemporaneous physiological data (FIG. 2B). Heart rate variability (HRV) 220 is an important physiological data for emotion change analysis and predictions. HRV is a measure of the variation in time between consecutive heartbeats, and it is associated with different emotional states. For example, a low HRV is associated with stress and anxiety, while a high HRV is associated with relaxation and positive emotions. Facial muscle activity 222 data can also be used for analysis and predictions of emotion changes as well as for detecting critical cues for emotion change analysis and predictions. Facial expressions are a powerful way to communicate emotions, and they are associated with different emotional states. For example, a neutral facial expression is associated with calm and relaxed emotions, while a frowning facial expression is associated with stress, frustration and negative emotions.

Body posture and gestures 224 data are essential components of non-verbal communication. They often convey emotions, intentions, and attitudes even in the absence of verbal cues. Analyzing these non-verbal signals can provide insights into a person's emotional state. Changes in body movement, such as shifts in posture, fidgeting, or pacing, can indicate emotional arousal or agitation. Analyzing body dynamics, such as gait patterns or hand movements, can provide additional cues for inferring emotional states. Body posture and gestures are interpreted within the context of the situation or environment. For example, crossed arms may indicate defensiveness or discomfort, but in some cultures, it could simply be a relaxed posture. Understanding the context is crucial for accurate interpretation of body language. Advances in sensor technologies, such as wearable devices or depth-sensing cameras, enable the capture of body posture and gesture information in real-time. These sensors can track movements and postures with high precision, providing rich data for emotion analysis and prediction.

Electroencephalogram (EEG) 226 data can also be used for analysis and predictions of emotion changes as well as for detecting critical cues for emotion change analysis and predictions. EEG is a technique that measures the electrical activity of the brain through electrodes placed on the scalp. Different emotional states are associated with distinct patterns of brain activity. EEG data can capture these patterns by measuring changes in electrical signals across different brain regions. For example, increased activity in the frontal cortex has been linked to positive emotions like happiness, while increased activity in the amygdala may indicate negative emotions like fear or sadness. Event-Related Potentials (ERPs) are specific patterns of brain activity that occur in response to stimuli, such as visual, auditory, or emotional cues. EEG can capture ERPs associated with emotional processing, providing insights into the neural mechanisms underlying emotions. EEG data can be analyzed in terms of different frequency bands, such as delta, theta, alpha, beta, and gamma waves. Changes in these frequency bands have been linked to different cognitive and emotional states. For example, increased alpha activity may indicate relaxation or a meditative state, while beta activity may indicate arousal or cognitive engagement.

Electrodermal Activity (EDA) 228 data can also be used for analysis and predictions of emotion changes as well as for detecting critical cues for emotion change analysis and predictions. Electrodermal activity, measures the electrical conductance of the skin, primarily influenced by sweat gland activity controlled by the sympathetic nervous system (SNS). EDA is particularly sensitive to changes in emotional arousal. Emotional arousal triggers the sympathetic nervous system, leading to increased sweat gland activity and subsequent changes in skin conductance level (SCL). Higher SCL values typically indicate greater emotional arousal, stress, or engagement with stimuli. Therefore, monitoring changes in SCL can provide real-time indicators of emotional arousal and intensity. EDA has been widely used in stress research and anxiety assessment. Stressful situations often elicit significant increases in SCL due to sympathetic activation.

Eye movements 230 data can also be used for analysis and predictions of emotion changes as well as for detecting critical cues for emotion change analysis and predictions. Eye movements, including fixations, saccades, and pupil dilation, can reflect underlying cognitive and emotional processes. Eye movements reflect where a person is directing their attention. Different emotions are associated with distinct patterns of attentional focus. For example, increased fixation on emotional stimuli (e.g., a sad face) may indicate emotional processing. Analyzing eye gaze patterns can provide insights into the salience of emotional cues and the allocation of attentional resources in response to emotional stimuli. Pupil size changes in response to cognitive and emotional stimuli. Pupil dilation is associated with arousal and cognitive processing load. Emotional stimuli that evoke strong emotional responses often lead to increased pupil dilation.

Monitoring changes in pupil size can provide real-time indicators of emotional arousal and engagement. The duration and frequency of fixations (periods of stable gaze) on specific regions of interest can reveal the intensity and duration of emotional processing. Longer fixations on emotional stimuli may indicate deeper emotional engagement. Analyzing fixation patterns across different emotional stimuli can identify which stimuli elicit the strongest emotional responses. Saccades (rapid eye movements between fixations) are associated with cognitive and attentional shifts. Changes in saccadic patterns in response to emotional stimuli can indicate shifts in attention and cognitive processing. For example, avoidance of emotional stimuli (e.g., averting gaze from a threatening image) may reflect emotion regulation strategies or discomfort with the stimulus. Respiration rate and depth 232 data can also be used for analysis and predictions of emotion changes as well as for detecting critical cues for emotion change analysis and predictions.

Emotions trigger physiological responses mediated by the autonomic nervous system (ANS), including changes in respiration patterns. The sympathetic nervous system (SNS) activation, associated with emotional arousal, often leads to changes in respiration rate and depth. Increased emotional arousal typically results in faster and shallower breathing patterns, reflecting the body's preparation for action. Conversely, relaxation and decreased arousal may lead to slower and deeper breathing. Respiration rate and depth are sensitive indicators of stress and anxiety. Emotional states such as stress, anxiety, or fear often lead to changes in breathing patterns. Blood pressure 234 is a physiological measure of the force exerted by the blood against the walls of the arteries as the heart pumps it through the body. Emotions trigger physiological responses mediated by the autonomic nervous system (ANS), including changes in heart rate and blood pressure. Emotional arousal typically leads to increases in blood pressure due to sympathetic nervous system activation. Monitoring changes in blood pressure can provide real-time indicators of emotional arousal and intensity. Emotional states such as stress, anxiety, or anger often led to increases in blood pressure. Emotional arousal can lead to changes in skin temperature 236 due to alterations in blood flow regulated by the sympathetic nervous system (SNS). Higher emotional arousal often results in vasoconstriction, reducing blood flow to the skin and causing a decrease in skin temperature.

Conversely, relaxation or decreased arousal can lead to vasodilation and an increase in skin temperature. Monitoring Emotional States: Skin temperature can be used as an indicator of emotional states, with lower temperatures indicating higher arousal and higher temperatures indicating relaxation. Continuous monitoring of skin temperature can provide insights into changes in emotional arousal over time. The physiological data can also include multi-signal composite metrics 238. Multi-signal composite metrics can include ML-derived composite metrics generated by the trained neural network. For example, a Stress Index or Resilience Score can serve as multi-signal composite physiological measures. These are calculated from a combination of key input signals such as heart rate variability (HRV), skin temperature, and respiratory rate, providing a holistic view of an individual's psychophysiological state. The physiological data can also include any other relevant physiological metrics not explicitly stated here. Pre-processing 240 helps to clean and prepare the input data for analysis, making it easier for models to extract meaningful insights and patterns. The specific pre-processing steps applied may vary depending on the task, domain, and characteristics of the input data. Pre-processing can refer to a series of steps and techniques applied to raw input data before they are used for modeling and analysis.

The goal of pre-processing is to clean, normalize, and transform the data into a format that is suitable for further analysis. The collected IESI 212 need to be pre-processed 242. As the IESI collected from pre-existing audio-video ERS can widely vary in terms of different IESI classification systems, and therefore can be represented in various formats, it can be useful to be mapped into a Multilayer Emotion Representation Framework (MERF) 244, before they are used for analysis and predictions using trained neural network. Different research communities and industries have used their own representations of emotions depending on the specific tasks and objectives they are addressing including for example Categorical Labels, wherein emotions are often represented as discrete categories, such as “happy,” “sad,” “angry,” etc. Another example of commonly used representations of emotions can include Continuous Valence-Arousal Space, wherein emotions can also be represented as points in a continuous two-dimensional space defined by valence (pleasantness) and arousal (intensity). Another example of commonly used representations of emotions can include Graph-Based Representations, wherein emotions and their relationships can be represented using graph-based structures, where nodes represent emotions and edges represent relationships between them.

Graph-based representations can capture complex interactions and dependencies between different emotional states. While it appears as of now there isn't currently a universally accepted representation of emotions, however the concept of Multilayer Emotion Representation Framework (MERF) involves developing its own unique representation. The representation of emotions used involves a Multilayer Emotion Representation Framework (MERF). This framework integrates categorical, dimensional, and relational (graph-based) components into a unified, multi-dimensional representation of emotions. The MERF is designed and developed to address both the universality and task-specific flexibility needed for diverse applications in emotion-related research and industries. Core components of MERF can include Categorical Layer (Primary Labels).

As emotions are first represented using discrete categorical labels, such as “happy,” “sad,” “angry,” etc., these labels serve as the fundamental building blocks, offering intuitive and interpretable classifications for tasks requiring explicit emotion identification. Another core component of MERF can include Dimensional Layer (Continuous Valence-Arousal Space). Each categorical label is mapped to a point in a continuous two-dimensional valence-arousal space, allowing for nuanced variations within each category.

For instance: “Happy” may span from low to high arousal depending on intensity (e.g., “content” vs. “ecstatic”) or “Sad” may include low-arousal (“melancholy”) and high-arousal (“distressed”) variations. Another core component of MERF can include Relational Layer (Graph-Based Representation) wherein emotions and their interactions are represented as nodes in a dynamic graph structure in which nodes correspond to specific emotions, including categorical labels and blended states (e.g., “bittersweet,” combining “happy” and “sad”), while edges represent relationships between emotions, including transitions (e.g.,“frustration”→“anger”) or co-occurrence patterns (e.g., “anxiety” often co-occurring with “fear”). On one hand, the innovations in MERF can include Hybrid Integration as MERF bridges the gap between discrete and continuous models by simultaneously leveraging the interpretability of categorical labels, the granularity of valence-arousal spaces, and the complexity of graph-based relationships. On the other hand, the innovations in MERF can include Universal and Task-Specific Flexibility including the categorical and dimensional layers provide universal foundations for emotion representation, the relational graph is customizable, allowing domain-specific modifications (e.g., mapping emotion transitions in emotional health contexts systems) as well as the Dynamic Adaptability as the graph-based layer can evolve over time, incorporating temporal and contextual changes.

Furthermore, the MERF can be visualized as a 3D structure. The X-Y plane can represent the valence-arousal space, mapping each emotion label to a continuous point while the Z-axis connects emotion nodes via edges, forming a layered graph showing transitions, co-occurrences, and dependencies. This visualization can offer a comprehensive view of emotional states, their intensity, and their interconnections in one coherent framework. In a similar way it can be useful that the collected further IESI 214 be pre-processed 246 and mapped into MERF (Multilayer Emotion Representation Framework) 248 before they are used for analysis and predictions using trained neural network. In much the same way the collected further data and emotional cues 216 need to be pre-processed 250. The next step for the IESI data and further data collected can be input embedding 260, 262, 264 and 266 which can refer to the process of representing input data sequences as dense, fixed-length vector representations before feeding them into the neural network. This can be followed by another step called integration of feature extraction and engineering 270.

The integration of feature extraction and feature engineering is the backbone of the multilayered machine learning for early detection, prediction, and proactive intervention in mental health care, transforming complex raw inputs into actionable intelligence. Feature extraction process can reduce the complexity of raw data while focusing on identifying the key attributes from raw multimodal data that represent the early detection, prediction, and proactive intervention in mental health care. This process can be followed by feature engineering process. Feature engineering goes beyond extraction by transforming, combining, or creating new features to improve the effectiveness of the machine learning model.

By embedding domain-specific knowledge, feature engineering process can enhance the predictive power of the features and can align them with the mental health care domain. It can bridge the gap between raw data and actionable insights. Feature engineering can optimize the extracted features for predictive and proactive mental health care. Together, feature extraction and feature engineering processes can enable the machine learning model to delve deeply into the complexities of human emotions and provide early detection, prediction, and proactive intervention in mental health care while ensuring the features are highly relevant. To provide some examples, feature engineering techniques can include Data Transformation. Data transformation techniques can include Normalization, Standardization, etc. To provide some examples, feature engineering techniques can include Feature Creation.

Feature creation can include Derived Features. Derived features can include creating new features from existing ones, e.g., combining “heart rate” and “skin conductance” into a “stress index.” Feature creation can include Interaction Features. Interaction features can include combining two or more features multiplicatively or additively to capture interactions (e.g., “voice pitch*speech tempo”). Feature creation can include Aggregated Features. Aggregated features can include summarizing temporal data using mean, variance, median, min/max, etc. (e.g., average HRV over a week). Feature creation can include Cumulative Features. Cumulative features can include tracking trends by summing or accumulating values over time (e.g., cumulative stress signals).

Feature creation can include Bin-Based Features. Bin-Based features can include grouping continuous variables into bins or categories (e.g., low/medium/high stress). To provide some examples, feature engineering techniques can include Dimensionality Reduction. Dimensionality reduction can include Principal Component Analysis (PCA). PCA can reduce data to its most important components while preserving variance. Dimensionality reduction can include t-SNE/UMAP. T-SNE/UMAP can visualize and reduce high-dimensional data into low-dimensional space for clustering or analysis. Dimensionality reduction can include Autoencoders. Autoencoders can include deep learning-based reduction by encoding features into a compressed representation. To provide some more examples, feature engineering techniques can include Temporal Feature Engineering. Temporal feature engineering can include Rolling/Averaged Features. Rolling/averaged features can include extracting rolling averages or variances over a time window (e.g., heart rate over the last 5 minutes).

Temporal feature engineering can include State Change Indicators wherein State Change Indicators can capture sudden transitions in time series (e.g., abrupt spikes in skin conductance). Temporal feature engineering can include Lag Features wherein Lag Features can incorporate values from previous time steps as features (e.g., past HRV trends affecting current predictions). To provide some more examples, feature engineering techniques can include Feature Selection. Feature selection can include Correlation Analysis. Correlation analysis can include selecting features that are highly correlated with the target variable. Feature selection can include LASSO or Ridge Regularization. LASSO or Ridge Regularization can include using penalties in regression to shrink irrelevant features toward zero. Feature selection can include Recursive Feature Elimination (RFE) wherein RFE can iteratively remove less important features based on model weights. Feature selection can include Mutual Information techniques to identify features that have the highest dependency on the target variable. To provide more examples of feature engineering techniques, Contextual Enrichment can be useful. Contextual Enrichment can include Baseline Comparisons.

Baseline comparisons can calculate differences or deviations from a patients-specific baseline (e.g., typical heart rate vs. current rate). Contextual Enrichment can include External Data Integration in a way that it can for example add context from external sources, such as environment, location, or time of day. Handling Missing Data, can also be a useful feature engineering technique. As an example, Imputation technique, can include filling missing values using techniques like mean, median, or interpolation. As another example Indicator Variables, can include adding flags for missing data to help the model learn patterns around it. To provide more examples of feature engineering techniques, Discretization and Binning can also be useful. This technique can include Quantile Binning wherein quantile binning can divide continuous variables into equal-sized bins (e.g., quartiles). Discretization and Binning can also include Custom Binning wherein it can create bins based on domain knowledge (e.g., stress levels categorized as “low,” “medium,” “high”). Scaling and Weighting can also be useful. An example technique can include Feature Scaling wherein it can adjust the magnitude of features to bring them into a comparable range (e.g., MinMaxScaler). Weighting Features can also be mentioned as another example wherein it can assign more importance to specific features based on domain knowledge. To provide more examples, feature engineering techniques can include Advanced Feature Engineering for Sequence Data. These techniques can include Fourier Transform, Wavelet Transform, Hidden Markov Models (HMMs), etc. Outlier Handling can also be mentioned as another example for feature engineering techniques.

Outlier handling can include Winsorization, clipping, Anomaly Flags, etc. While various examples for feature engineering techniques were provided, however, the examples provided should not limit the spirit and scope of the present invention; but rather should be understood in the broadest sense allowable by law. In a similar way, the input embeddings are used for the feature extraction process 272, 274, 276 wherein the model learns representations that capture the contextual information and semantic relationships within the sequence data which will then be followed by feature engineering process.

Multi-task learning 282, 284, 286 is a technique used in sequence modeling that allows a model to learn multiple tasks at the same time. The idea behind multi-task learning is that a model trained on multiple tasks can learn shared representations or features that can be useful for all the tasks, and this can improve the performance of the model on each task. This can result in models that capture richer representations of the input sequence and achieve better performance on unseen data. It can also lead to faster convergence during the optimization process. Among various approaches multi-task learning can include multi-task learning with attention wherein attention mechanisms can be used to selectively focus on different parts of the input sequence for different tasks wherein this approach allows the model to learn different representations for each task. During multi-task learning process, the parameters of the neural network, including the input embeddings and the weights, are optimized using backpropagation through time (BPTT) or other optimization algorithms.

The objective is to minimize a loss function that measures the discrepancy between the predicted outputs and the true labels or targets associated with the input sequences. The sequence modeling system can include neural networks, where the neural network can be part of machine learning techniques. The sequence modeling system can include attention mechanisms. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks. An example of sequence model computing methods leveraging attention mechanisms can involve Long Short-Term Memory (LSTM) networks with attention models.

LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts. In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data. As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights.

Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions.

As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation. Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. The learning can facilitate early detection, prediction, and proactive interventions 290 in mental health care. A multilayered machine-trained sequence modeling system learns trained weights using the collected series of IESI over time, wherein the learning can facilitate analysis using trained neural network 292.

Further contemporaneous IESI based on audio-only and video-only can be collected separately into the computing device. The computing system can learn trained weights using the further IESI, wherein the trained weights are trained through multi-task learning. The computing device can analyze the further IESI using trained weights to perform further analysis including consistency examination 294, as well as to facilitate early detection, prediction, and proactive intervention in mental health care, based on the further IESI. Further data including emotional cues are collected into a second computing device wherein the computing device can learn trained weights using the further data wherein the trained weights are trained through multi-task learning. The computing device can analyze the further data using trained weights to perform further analysis including analysis of critical cues 296 to facilitate early detection, prediction, and proactive intervention in mental health care based on the further data.

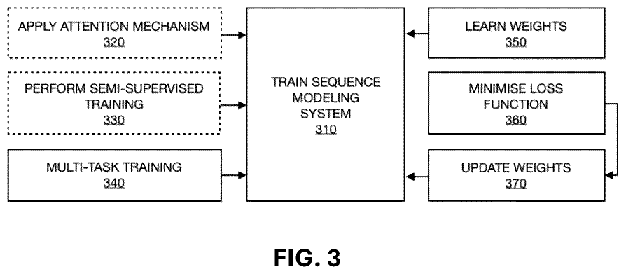

Turning to FIG. 3, Flow 300 is a flow diagram for training. In some implementations, the method may include training sequence modeling system 310, where sequence modeling system learns weights 350 through multi-task training 340; minimizing loss function 360; and updating weights 370. In some implementations, the method may include integration of steps of applying attention mechanism 320, and/or performing semi-supervised training 330.

A schematic representation of the training components is shown utilized within the sequence modeling framework. The central element, train sequence modeling system 310, functions as the core computational module where various machine learning processes are integrated to analyze IESI. Inputs to train sequence modeling system 310 include apply attention mechanism 320, which assigns varying levels of relevance to data inputs during processing. This mechanism ensures that the system emphasizes the most pertinent data points, enhancing predictive accuracy. Additionally, perform semi-supervised training 330 provides the integration of both labeled and unlabeled data, facilitating robust learning under diverse data conditions. Multi-task training 340 enables simultaneous execution of multiple learning tasks within the system. This concurrent processing capability enhances the efficiency and adaptability of the model, allowing for comprehensive emotional data analysis.

Outputs from train sequence modeling system 310 connect to several essential processes. Learn weights 350 involves the adjustment of parameters within the model to optimize performance based on input data. Minimize loss function 360 focuses on reducing discrepancies between predicted and actual outcomes, thus enhancing the model's fidelity. Update weights 370 represents the ongoing refinement of the model through iterative adjustments of previously established weight values. This iterative process supports continuous improvement in the predictive capabilities of the sequence modeling framework, ensuring responsiveness to new data inputs and changing emotional dynamics.

Analysis and predictions of emotion changes can involve training a sequence modeling system 310. The sequence modeling system can include neural networks, where the neural network can be part of machine learning techniques. The sequence modeling system can include attention mechanisms 320. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks. Sequence modeling systems can be trained using supervised, semi-supervised, or unsupervised learning approaches, depending on the availability and nature of labeled and unlabeled data. Semi-supervised learning 330 is a machine learning technique that utilizes a combination of labeled and unlabeled data to train models. This approach offers several benefits compared to traditional supervised learning. In supervised learning, the model is trained on a dataset that includes input-output pairs, where the output is the correct label or value for the input. In contrast, semi-supervised learning uses a dataset that includes some input-output pairs (labeled data) and some inputs without the corresponding outputs (unlabeled data).

The main idea behind semi-supervised learning is to use the unlabeled data to improve the generalization ability of the model by leveraging the additional information that it provides. The model is trained on the labeled data to learn the mapping between inputs and outputs, and then it is fine-tuned on the unlabeled data to improve its accuracy. Semi-supervised learning can improve the performance of models by leveraging the additional information from unlabeled data. This can be particularly useful in cases where labeled data is scarce or expensive to obtain. By using unlabeled data, semi-supervised learning can reduce the amount of annotation required to train a model. This can be more cost-effective and efficient than relying solely on labeled data. Semi-supervised learning can be useful for handling complex and high-dimensional data. It can be also used to handle imbalanced datasets, where the proportion of samples in different classes is unbalanced. Semi-supervised learning can be used to handle datasets with noisy labels or missing data, by using the unlabeled data to help the model distinguish between good and bad labels. However, the performance of semi-supervised learning is highly dependent on the quality and quantity of the unlabeled data.

Multi-task training 340 is a technique used in sequence modeling that allows a model to learn multiple tasks at the same time. This approach is also known as simultaneously learning. The idea behind multi-task learning is that a model trained on multiple tasks can learn shared representations or features that can be useful for all the tasks, and this can improve the performance of the model on each task. This can result in models that capture richer representations of the input sequence and achieve better performance on unseen data. It can also lead to faster convergence during the optimization process.

Among various approaches training simultaneously can include multi-task learning with attention wherein attention mechanisms can be used to selectively focus on different parts of the input sequence for different tasks wherein this approach allows the model to learn different representations for each task. An example of sequence model computing methods leveraging attention mechanisms can involve Long Short-Term Memory (LSTM) networks with attention models. LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts.

In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction. By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data.

As an example, in LSTM networks with attention models using semi-supervised machine learning, the process of learning weights 350 can involve leveraging both labeled and unlabeled data to improve model performance, wherein the process can involve learning and training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network. The model can make predictions based on the current weights. The process of minimizing loss function 360 is at the core of machine learning and can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions.

As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by updating the weights 370, wherein, the weights of the network can be adjusted in the direction that minimizes the loss function, based on the gradients computed during backpropagation. Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. The learning can facilitate analysis and predictions of emotion changes.

In some implementations, a method is provided including steps of training a sequence-modeling system configured to learn model parameters through multi-task learning, comprising minimizing at least one loss function and updating the model parameters based on said loss minimization. In some implementations, the method further includes steps of applying one or more attention mechanisms within the sequence-modeling system; and/or performing semi-supervised learning to further refine the model parameters using a combination of labeled and unlabeled data.

Also presented in this disclosure includes a system and methods for machine-trained analysis and predictions of emotion changes for early detection, prediction, and proactive intervention in mental health care. Flow 400 is a workflow encompassing data collection, computing system and output results. In some implementations, the method may include steps of capturing different categories of data 430 from an individual 410, including: collecting “further data” 456 through pre-existing sensors 420, collecting IESI 452 through pre-existing ERS 440; collecting “further IESI” 454 through pre-existing ERS 440; inputting the captured input sequence into a machine-learning computing system 460 configured to perform sequence-based analytical processing; providing machine-learning-based analytical outputs configured to analysis of early detection, prediction, and proactive intervention 470; and outputting the results to the real-time output unit and/or user interaction 480.

In some implementations, the method may include steps of capturing multiple categories of data from an individual, comprising: collecting additional data (“further data”) obtained from one or more pre-existing sensors; collecting Instant Emotional State Information (IESI) obtained from one or multiple pre-existing Emotion Recognition Systems (ERS); collecting additional contemporaneous IESI (“further IESI”) obtained from the pre-existing ERS; providing the plurality of captured data as input sequences to a machine-learning computing system; processing the input sequences using at least one trained model configured to perform early detection, prediction, and proactive-intervention analysis; and outputting the resulting early detections, predictions, or alerts to a real-time output interface and/or a user-interaction system.

The emotional state information may come from individuals 410 such as users, patients, clients, and others who are interested in or seeking to improve their mental health. Emotions coming from an individual can be expressed through a variety of verbal and nonverbal behaviors and can provide valuable information about a person's emotion dynamics.

Using specialized sensors 420 various physiological and behavioral data can be captured. For example, audio data can be captured using microphones, digital audio interfaces, USB microphones, clinical-grade biomedical audio acquisition systems integrated into observation rooms and psychophysiological monitoring setups, or any other similar sensors and equipment used in clinical settings. Video data can be captured through standard cameras or camcorders, as well as specialized medical imaging units such as patient observation cameras, endoscopic systems, and telehealth, telepsychiatry video platforms, or any other similar sensors and equipment used in clinical settings. Heart rate variability (HRV) data can be captured using electrocardiography (ECG) or photoplethysmography (PPG) sensors embedded in clinical monitoring systems, pulse oximeters, Holter recorders for continuous cardiovascular assessment, or any other similar sensors and equipment used in clinical settings. Facial muscle activity can be captured using electromyography (EMG) or facial EMG sensors, 3D facial motion capture systems, computer vision-based facial analysis tools employed in neurology, psychiatry, and rehabilitation contexts, or any other similar sensors and equipment used in clinical settings. Body posture and gesture data can be captured using motion capture systems, inertial measurement unit (IMU)-based wearable sensors, pressure-sensitive platforms, depth cameras within gait analysis and rehabilitation facilities, or any other similar sensors and equipment used in clinical settings.

Electroencephalogram (EEG) data can be captured using wired or wireless EEG caps, dry-electrode systems, hybrid EEG-fMRI/MEG configurations commonly used in neurology departments and sleep laboratories, or any other similar sensors and equipment used in clinical settings. Electrodermal activity (EDA) data can be captured through skin conductance sensors or clinical-grade EDA modules incorporated into psychophysiological, stress assessment, and biofeedback systems, or any other similar sensors and equipment used in clinical settings. Eye movement data can be captured using infrared eye-tracking systems, electrooculography (EOG) sensors, clinical video-oculography and vestibular testing platforms for neurodiagnostic evaluation, or any other similar sensors and equipment used in clinical settings. Respiration rate and depth data can be captured using respiratory belts, pneumography sensors, bioimpedance modules, capnography units integrated into intensive care and anesthesia monitoring systems, or any other similar sensors and equipment used in clinical settings.

Blood pressure data can be captured via sphygmomanometers, digital or ambulatory monitors, photoplethysmography-based sensors embedded in multi-parameter vital signs monitors, or any other similar sensors and equipment used in clinical settings. Skin temperature data can be captured using infrared thermometers, thermal imaging cameras, thermistor-based temperature sensors incorporated into patient monitoring and critical care systems, or any other similar sensors and equipment used in clinical settings. Computing device is configured to collect a plurality of contemporaneous IESI 452 over time, from an individual, from one or multiple pre-existing audio-video ERS 440, wherein the pre-existing audio-video ERS can be additionally benefited from further modalities including various contemporaneous physiological data.

A multilayered machine learning computing system learns trained weights using the collected series of IESI over time. Further IESI can be collected 454 into the computing device. The further IESI can include contemporaneous IESI based on audio-only and contemporaneous IESI based on video-only which each can be collected separately into the computing device in which, the computing device learns trained weights using the further IESI, wherein the trained weights are trained through multi-task learning. The computing device can analyze the further IESI using trained weights to perform further analysis including consistency examination, as well as to facilitate early detection, prediction, and proactive intervention in mental health care, based on the further IESI. Further data are collected 456, into a second computing device wherein the computing device can learn trained weights using the further data wherein the trained weights are trained through multi-task learning.

The second computing device can analyze the further data using trained weights to provide further information including emotional cues to facilitate early detection, prediction, and proactive intervention in mental health care, based on the further data. Further data can include various contemporaneous physiological data, such as contemporaneous heart rate variability data, facial muscle activity, skin temperature, blood pressure, respiration rate and depth, eye movement, electroencephalogram (EEG), electrodermal activity (EDA), body posture, and gestures information, multi-signal composite metrics, or any other applicable physiological data.

In embodiments, the further data can include one or more of contemporaneous physiological data. In embodiments, the computing device and the second computing device are a same computing device. The input sequence 450 collected needs to undergo preprocessing before being fed into the machine learning computing system. Analyze using trained neural network can include multilayered machine learning computing system 460, wherein the computing system can include neural networks, where the neural network can be part of machine learning techniques.

The sequence modeling system can include attention mechanisms. In sequence models with attention mechanisms, the model learns a set of weights that are used to calculate the attention scores for each element in the input sequence. These weights are typically trained during the model's training phase by minimizing a loss function, such as cross-entropy, using an optimization algorithm like stochastic gradient descent. The attention scores are then used to weight the importance of each element in the input sequence when generating the output sequence. This allows the model to focus on the most relevant parts of the input when making predictions, which can improve the model's performance on certain tasks.

An example of sequence model computing methods leveraging attention mechanisms can involve Long Short-Term Memory (LSTM) networks with attention models. LSTM networks are a type of recurrent neural network (RNN) used to capture long-range dependencies in sequences. They are equipped with memory cells and gating mechanisms that allow them to effectively process and remember information over long sequences. Attention mechanisms, on the other hand, enable the model to focus on specific parts of the input sequence while making predictions. Instead of treating all parts of the sequence equally, attention mechanisms assign different weights to different parts based on their relevance to the current prediction. This allows the model to selectively attend to important information and ignore irrelevant parts. In LSTM networks with attention models, the LSTM layer processes the input sequence and captures its temporal dependencies. The output of the LSTM layer is then passed through an attention mechanism, which dynamically computes attention weights for each time step in the sequence. These attention weights indicate the importance of each time step in making the final prediction.

By combining LSTM networks with attention mechanisms, the model can effectively capture long-range dependencies in sequential data while selectively focusing on relevant information. Sequence modeling systems can be trained using supervised, semi-supervised, or unsupervised learning approaches, depending on the availability and nature of labeled and unlabeled data. As an example, in LSTM networks with attention models using semi-supervised machine learning, the process of learning and training weights can involve leveraging both labeled and unlabeled data to improve model performance, wherein the process can involve training the weights of the network to effectively capture the relationships between input sequences and their corresponding outputs. This training process can involve various steps including initialization, wherein the weights of the LSTM and attention layers can be initialized randomly or using pre-trained weights. This can be followed by forward pass, wherein during training, input sequences, along with their corresponding labels (if available), are fed into the network.

The model can make predictions based on the current weights. Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions. As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation.

Subsequently, the process of forward pass, loss calculation, backpropagation, and weights update can be repeated iteratively over multiple epochs until the model converges to a satisfactory solution. During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data.

In the example of LSTM networks with attention models the model can maintain an internal state or memory cell that captures the context of the input sequence up to the current position. After processing the input sequence up to a certain point, the model can generate a probability distribution for the next emotion or token. In the LSTMs example, this can typically be achieved by for example applying a softmax activation function to the output of the model's final layer, producing a probability distribution over all possible emotions or tokens. Once the probability distribution is obtained, the next emotion or token can be generated using various ways including sampling, argmax decoding, etc.

Sampling can involve randomly selecting an emotion according to the probability distribution, which can introduce randomness and diversity into the predictions. Argmax decoding can involve selecting the emotion with the highest probability, resulting in deterministic predictions but potentially limiting diversity.

During training, input sequences, along with their corresponding labels (if available), are fed into the network. The model can make predictions based on the current weights. Loss functions can be used to quantify the difference between the predicted outputs and the actual labels. For labeled data, the loss function compares the predicted outputs with the true labels. For unlabeled data, the loss function may incorporate additional regularization terms to encourage smoothness or consistency in predictions. As another step, in backpropagation the error signal can be propagated backward through the network using gradient descent optimization algorithms, such as stochastic gradient descent (SGD) or Adam, etc. This can involve calculating the gradients of the loss function with respect to each weight in the network. This can then be followed by weights update, wherein, the weights of the network can be adjusted in the direction that minimizes the loss function, based on the gradients computed during backpropagation. Subsequently, the process of forward pass, loss calculation, backpropagation, and weights update can be repeated iteratively over multiple epochs until the model converges to a satisfactory solution.

During training, the LSTM network can learn to capture long-term dependencies in the input sequences, while the attention mechanism can learn to dynamically focus on relevant parts of the input. By adjusting the weights of both the LSTM and attention layers, the model can improve its ability to make accurate predictions on new, unseen data. The objective is to minimize a loss function that measures the discrepancy between the predicted outputs and the true labels or targets associated with the input sequences wherein the learning can facilitate analysis of early detection, prediction, and proactive intervention 470 in mental health care. Real-time output unit and user interactions 480 can involve the immediate presentation of analysis and predictions of emotion changes for early detection, prediction, and proactive intervention in mental health care.

Methods for detecting and predicting emotion changes for early detection, prediction, and proactive intervention in mental health care, are provided. In some implementations, the methods may include steps of real-time monitoring of one or more emotional dynamics; deriving one or more insights based on the one or more emotional dynamics; generating one or more predictive analytics from the monitored one or more emotional dynamics and derived one or more insights; identifying one or more target emotional indicators, trends, patterns, and risks based on the one or more detection and predictive analytics; predicting one or more target emotional indicators, trends, patterns, and risks based on the one or more predictive analytics; generating at least one alert or flag signal based on identified or predicted one or more target emotional indicators, trends, patterns, and risks; generating one or more proactive intervention recommendations; generating one or more care-related guidance or recommendations; generating one or more personalized or adaptive care plans; constructing one or more real-time analytical summaries based on the generated one or more proactive intervention recommendations, one or more care-related guidance or recommendations, or one or more personalized or adaptive care plans; displaying a visualization of analysis results of the one or more real-time analytical summaries; and exporting or transmitting the analysis results in one or more data formats.

In some implementations, the methods may include steps of providing analysis results through a real-time output subsystem and/or user-interaction interface, the analysis results comprising one or more of: real-time monitoring of emotional dynamics; real-time analytical outputs and derived insights; predictive analytics generated from the captured and processed data; identification of target emotional indicators, trends, patterns, and risks; prediction of target emotional indicators, trends, patterns, and risks; generation of alert or flag signals based on detected or predicted conditions; generation of proactive intervention recommendations; generation of care-related guidance or recommendations; generation of personalized or adaptive care plans; construction and presentation of real-time analytical summaries; visualization of analysis results through one or more display modalities; exporting or transmitting the analysis results in one or more data formats.

Flow 500 is a flow diagram for output unit and user interactions. Real-time output units and user interactions 510 involve the immediate presentation of analysis results and predictions regarding emotion changes for early detection, prediction, and proactive intervention in mental health care, utilizing trained neural networks and the disclosed machine learning system and methods. These outputs form the basis for engaging with users, enabling timely responses and personalized interventions. Real-time output unit and user interactions can include providing real-time monitoring of emotional dynamics, real-time analytics and insights, predictive analytics, providing identification of target emotional indicators, trends, patterns, and risks, providing prediction of target emotional indicators, trends, patterns, and risks, providing flagging system, proactive intervention, care recommendations, and personalized care plans, etc.

Real-time monitoring of emotion dynamics 520 can include continuous tracking of how emotions fluctuate over time, revealing their patterns, speed, variability, and transitions (e.g., steady rises in sadness or rapid shifts in affect), whereas absolute emotions identify and categorize specific emotional states at a single time point without considering their contextual or temporal evolution; the study of emotion dynamics, mediated by physiological arousal, cognitive appraisals, and neural mechanisms such as heart rate, respiratory changes, skin conductance, and muscle activity, provides deeper insight into temporal processes, contextual influences, and the adaptive role of emotions compared to static analysis of absolute states. Monitoring of emotion dynamics seeks to uncover the processes, underlying mechanisms triggers, and patterns of emotional experiences over time.

This process which is discussed in more details in the flow 200, can include pre-processing of the input data, mapping into MERF (Multilayer Emotion Representation Framework), undergoing embedding processes, followed by feature extraction, where the trained neural network converts the raw emotion signals into descriptive features used for continuous multimodal emotion dynamics tracking with physiological correlates. Real-time output unit and user interactions can include real-time analytics and insights 522. Providing real-time analytics and insights is the foundation for delivering instant insights and responses to caregivers. In the mental health care domain, leveraging machine learning and trained neural network for real-time analytics involves the seamless processing and analysis of continuous emotional and behavioral data.

Real-time analytics can provide caregivers an immediate emotional insight to gain real-time awareness of emotional changes, fostering better decision-making and timely interventions. Moreover, caregivers receive an ongoing analysis of patient's mental health indicators, trends, patterns, and emerging risks enabling proactive mental well-being management. Providing real-time analytics using trained neural network can enable the mental health platform to effectively respond to the complexities of human emotions by acting as the foundation for both predictive and proactive mental health care. Real-time output unit and user interactions can include providing predictive analytics 524. In the mental health care domain providing predictive analytics is the foundation for proactive care and proactive intervention. Leveraging machine learning and trained neural network, predictive analytics involves forecasting future emotional indicators, trends, patterns, and identify potential risks, and proactively address emerging issues in mental health care. Predictive analytics can provide proactive mental health management. By identifying risks early, predictive analytics can provide caregivers take preventive measures to avoid emotional crises. Predictive analytics can provide precision in emotional interventions.

Predictive analytics can ensure that interventions are timely and highly relevant, improving their effectiveness. Predictive analytics can also provide reduction in healthcare costs. Early detection and intervention can reduce the need for costly treatments and hospitalizations associated with advanced mental health conditions. Predictive insights can empower caregivers with actionable knowledge, helping them stay ahead of potential mental challenges. Predictive analytics, using machine learning powered by trained neural networks, is a cornerstone of an ML-driven mental health care platform. By forecasting emotional indicators, trends, patterns, and identifying risks, it can enable proactive interventions, tailored support, and better mental health management. This capability can transform mental care into a forward-looking, data-driven practice, creating lasting impacts for individuals and the broader healthcare system. Real-time output unit and user interactions can include providing identification of target emotional indicators, trends, patterns, and risks 526.

Target emotional indicators, trends, patterns, and risks can encompass a wide range of clinically and psychologically meaningful outcomes that are valuable for detection and monitoring in mental health care. These outcomes can closely relate to the presence or progression of symptoms and disorders within mental health contexts. Examples of symptom-related indicators, trends, patterns, and risks can include the early detection or identification of symptoms such as depressed mood, generalized anxiety, panic symptoms, and similar manifestations. Each detected symptom can be accompanied by an associated confidence score, severity level, and information about whether it is stable, improving, or worsening over time. The model may also generate respective hourly, daily, weekly, or monthly symptom trajectories to illustrate these changes dynamically.

Examples of disorder-related indicators, trends, patterns, and risks can include the early detection or identification of mental health disorders such as Major Depressive Disorder (single or recurrent episode, moderate severity), Generalized Anxiety Disorder, or Persistent Depressive Disorder (Dysthymia). These detections can include associated confidence scores, severity levels, and temporal dynamics derived from ongoing analysis. Each of the above indicators, trends, patterns, and risks is mapped to its corresponding extracted feature within the machine learning framework. These features are processed and interpreted by trained neural network model, enabling accurate, data-driven insights into patients'emotional indicators, trends, patterns, and risks. The mechanism of identification of target emotional indicators, trends, patterns, and risks in the context of sequence modeling can involve detecting regularities, structures, or recurring motifs within input sequence data. Before identifying indicators, trends, patterns, and risks, the raw sequence data often needs to be preprocessed and transformed into a suitable format in which the process may involve extracting features. Sequences of emotions are typically tokenized and converted into numerical representations using techniques like input embeddings. The preprocessed sequences can then be fed into a sequence model for analysis. The sequence model then can learn to capture the underlying patterns in the data by iteratively processing the input sequences. Real-time output unit and user interactions can include providing prediction of target emotional indicators, trends, patterns, and risks 528.

Prediction of target emotional indicators, trends, patterns, and risks can involve the application of machine learning techniques designed for Next Emotion Prediction (NEP). Using trained neural network model, the system analyzes current and historical emotional data to anticipate an individual's future emotional data. This predictive capability enables the trained neural network model, to forecast potential emotional indicators, trends, patterns, and emerging risks before they manifest. The Next Emotion Prediction process can generate one or multiple possible future scenarios, each associated with a probability or confidence level, allowing for more informed and proactive mental health interventions. Target emotional indicators, trends, patterns, and risks can encompass a wide range of clinically and psychologically meaningful outcomes that are valuable for predictive analysis in mental health care. These outcomes can relate to the onset, progression, or recurrence of symptoms and disorders within mental health contexts. Examples of symptom-related indicators, trends, patterns, and risks can include the prediction of symptoms such as depressed mood, generalized anxiety, panic symptoms, and similar manifestations.

Each predicted symptom is accompanied by an associated confidence score and severity risk level, providing interpretable, data-driven estimates of likelihood and intensity. Examples of disorder-related indicators, trends, patterns, and risks can include the prediction of mental health disorders such as posttraumatic stress disorder (PTSD), Schizophrenia, Bipolar I Disorder (Current or Most Recent Episode, Manic), obsessive-compulsive disorder (OCD), and similar manifestations. These predictions can include associated confidence scores and severity risk assessments that quantify the model's certainty and the potential clinical impact.

Each of the indicators, trends, patterns, and risks is mapped to its corresponding extracted feature within the machine learning framework. These features are then processed and interpreted by trained neural network model, enabling precise, data-driven insights into patients'emotional indicators, trends, patterns, and risks. The mechanism of next emotions prediction in the context of sequence modeling can involve predicting the next emotion or token in a sequence of emotions or tokens given the preceding context. In other words, it involves prediction of the most likely emotions that follow a given sequence of emotions, given the context provided by those emotions. This task can be addressed using various sequence modeling techniques, including LSTMs with attention models. The input to the model can consist of a sequence of emotions or tokens, represented as numerical vectors. Each emotion or token can be typically converted into a high-dimensional vector using techniques like input embeddings. The input sequence can then fed into the model, and the model can process each emotion or token one at a time.

Real-time output unit and user interactions can include providing flagging system 530. The mechanism of a flagging system can be used to highlight or flag specific indicators, trends, patterns or any potential risks or opportunities that are of interest or concern. The purpose of the flagging system can be to draw attention to those indicators, trends, patterns or potential risks or opportunities so that appropriate actions can be taken in advance. The flagging system for predictive analytics using sequence modeling system can analyze data in real-time and raise a flag or alert as soon as a potential issues or opportunities are predicted. Flagging rules can be defined to determine when to raise a flag based on the identified emotional indicators, trends, patterns or potential risks or opportunities. These rules may specify conditions under which certain indicators, trends, patterns or potential risks or opportunities should be flagged, such as exceeding predefined thresholds, or indicating potential risks or opportunities.

A flagging mechanism is implemented to automatically raise flags when the predefined conditions specified by the flagging rules are met. Flags can be generated in real-time as new data becomes available. Flagged indicators, trends, patterns or potential risks or opportunities can be then visualized and reported accordingly to take proactive measures to address them before they happen and become a problem. Real-time output unit and user interactions can include providing proactive intervention 532. Traditional mental health care is largely reactive—clinicians typically intervene after symptoms have worsened or crises have already occurred. In contrast, proactive intervention focuses on anticipating and addressing risks early, before acute symptoms manifest or escalate.

By continuously analyzing real-time and historical emotional data, trained neural network model can detect emerging risks, predict future scenarios, trigger automated flagging systems, deliver timely interventions, and generate personalized care recommendations and plans. This proactive approach can empower caregivers, helping to reduce emotional crises and enhance the overall effectiveness of mental health care. Proactive intervention can lessen the severity of mental health challenges, improve treatment outcomes, and lower healthcare costs by reducing the need for intensive therapy sessions or emergency medical interventions. Proactive intervention also enables tailored, user-centric care, as recommendations and actions are personalized to each individual's unique emotional patterns, preferences, and behavioral history. In practice, proactive intervention can anticipate symptom escalation before it occurs. Proactive intervention can prioritize high-risk patients within clinical caseloads. Proactive intervention can dynamically tailor interventions, such as adjusting medication, increasing therapy frequency, or recommending targeted coping strategies. When the system detects a rising risk, proactive intervention can take various forms depending on the severity and context, such as: providing clinician alerts (enabling early outreach before relapse), providing AI-generated emotional regulation support (offering immediate self-guided interventions), providing treatment adjustment recommendations (assisting clinicians in fine-tuning care dynamically), providing crisis prevention protocols (preventing escalation to emergency situations).

By addressing subtle emotional fluctuations early, proactive interventions can stabilize emotional trajectories, reduce the likelihood of acute episodes, hospitalizations, or dropouts, and enhance long-term patient well-being. Each proactive intervention and its outcome (e.g., improvement or no change) provides new labeled data back to the model. This creates a continuous learning loop: Detection→Prediction→Intervention→Outcome→Learning→Improved Model Accuracy. Over time, the trained neural network can learn which interventions are most effective for which profiles, leading to highly personalized and context-aware care recommendations. This supports precision mental health care, where interventions are dynamically adapted to each individual's unique emotional and physiological indicators, trends, patterns, and risks.

Real-time output unit and user interactions can include providing care recommendations. Providing care recommendations can refer to generating data-driven, personalized, and context-aware suggestions that guide clinicians in optimizing mental health care. These recommendations are not generic “tips,” but intelligent, adaptive outputs produced by the trained neural network. They are grounded in continuous emotional monitoring, predictive analytics, and contextual interpretation of multimodal data. in other words, care recommendations can translate analytic intelligence into actionable clinical and behavioral guidance. Care recommendations are essential because they bridge the gap between data insight and clinical action. Care recommendations can support mental health care by enhancing clinical decision-making by providing clinicians with AI-assisted insights on care optimizations, what to adjust or prioritize in treatment. Care recommendations can support mental health care by helping clinicians with standardize decision support when clinician availability is limited. Care recommendations can support mental health care by offering personalized care plans. Care recommendations can support mental health care by identifying warning patterns early and suggest preventive interventions before emotional crises escalate. Care recommendations can support mental health care by encouraging adaptive coping behaviors that stabilize emotional trajectories. Care recommendations can support mental health care by helping extend clinician reach by providing automated, evidence-informed recommendations to large patient populations. Care recommendations can support mental health care by supporting scalable, data-driven care.

Real-time output unit and user interactions can include providing personalized care plans. Personalized care plans include ML-generated personalized care plans wherein, ML-generated personalized care plans are personalized care plans generated by the trained neural network. ML-generated personalized care plans can include pre-authorization ML-generated personalized care plans wherein pre-authorization ML-generated personalized care plans can include ML-generated, personalized, evidence-informed, and patient-specific treatment roadmap that can be automatically created before a clinician formally authorizes or modifies it. Personalized care plans can include multidisciplinary or integrated care. Care plans can include clinical formulation, including conceptual/diagnostic reasoning. Care plans can include treatment plans. Care plans can include psychiatric management plans including medication and symptom management. ML-generated personalized care plans can integrate real-time emotional analytics, longitudinal behavioral data, and predictive modeling results to recommend initial or adaptive care strategies, while the clinicians can remain the ultimate decision-maker.

Traditional care planning in mental health usually relies on periodic assessments and manual interpretation of subjective data. This can delay interventions and reduce personalization. By contrast, the ML-generated personalized care plan, can provide immediate plan generation based on live emotional and physiological insights. The ML-generated personalized care plan can provide clinically interpretable recommendations for treatment. The ML-generated personalized care plan can provide continuous plan adaptation as new data arrives (e.g., emotional dynamics, symptom changes, compliance signals). The ML-generated personalized care plan can provide pre-authorization workflows—clinicians can review, approve, or edit ML-suggested plans before activation. This can bridge data analytics with clinical decision support, ensuring care plans evolve with patient needs while preserving medical oversight.

The benefits of ML-generated personalized care plans are multifaceted. ML-generated personalized care plans can enable immediate care plan generation as soon as a risk is detected, ensuring that early intervention can occur without delay. ML-generated personalized care plans also can provide tailored interventions that align with each patient's unique emotional, physiological, and behavioral profile, enhancing personalization and treatment relevance. Moreover, ML-generated personalized care plans can reduce the manual planning workload for clinicians by automating much of the data analysis and preliminary care design process, allowing practitioners to focus on clinical judgment and patient engagement. As emotional and behavioral data evolve over time, the system can dynamically update ML-generated personalized care plan, ensuring that recommendations remain responsive to the patient's changing condition. Additionally, ML-generated personalized care plans can enable proactive and scalable planning across large populations, supporting healthcare systems in delivering consistent, data-informed, and adaptive mental health care at scale.

Build and display real-time analytics summary 540 alongside visualizing real-time analytics summary 542 can involve presenting data insights in a clear, intuitive, and actionable manner. Effectively visualizing these data can include, for instance, the development of interactive dashboards capable of engaging users through features such as a detection module, prediction module, pre-auth care plan module, monitoring module, flagging system module, and urgent intervention module, in real-time. These dashboards can utilize charts, graphs, and gauges to visualize data. Additionally, integrating filters and drill-down capabilities can enable users to explore data across various levels of granularity.

Exporting data in multiple formats 544 can involve converting the output data into various file types or structures to accommodate different use cases and applications. Some common file types can include for example CSV (Comma-Separated Values) format, where each row represents a data point or observation, and columns represent different attributes or features. CSV files are widely supported by spreadsheet software and can be easily imported into data analysis tools for further processing. Another file type example can include JSON (JavaScript Object Notation) format, representing structured data as key-value pairs or nested objects. JSON files are commonly used for exchanging data between web services and applications and are easily readable by both humans and machines.

Another file type example can include XML (eXtensible Markup Language) documents, using tags to denote hierarchical relationships between elements. XML is suitable for representing complex data structures and is commonly used for data interchange in web services and document formats. Another file type example can include Excel Spreadsheets (.xlsx) containing the results, with each sheet representing a different subset of data or analysis. Excel files provide a familiar interface for viewing and analyzing tabular data and support various formatting options and formulas. Another file type example can include Text Files or plain text files, with each line representing a data point or record. Text files are lightweight and easily readable, making them suitable for exporting large datasets or logs. Another file type example can include Relational database management system (RDBMS) such as MySQL, PostgreSQL, or SQLite.

Exporting results to a database allows for efficient storage, retrieval, and querying of structured data, supporting complex data relationships and transactions. Another file type example can include Image Files for visual representation of patterns or outputs, such as graphs, charts, or heatmaps. Image files are suitable for exporting visualizations generated by the sequence model, enabling easy sharing and integration into reports or presentations. Another file type example can include custom file formats or data structures tailored to specific requirements or downstream applications. Custom formats can accommodate specialized data needs and may include metadata, annotations, or additional context relevant to the results. By exporting data results in multiple formats, a sequence modeling system ensures compatibility with diverse data analysis tools, visualization platforms, and downstream applications, enabling seamless integration and utilization of the model outputs across various domains and workflows.

In an aspect, the hardware infrastructure may include one or more of processing units configured to execute ML, analytical, and sequence-modeling operations; memory resources configured to store intermediate computations, model parameters, and input data; data-storage systems configured to retain training data, learned representations, and system outputs; networking components configured to enable communication between sensors, processing modules, and external systems.

In some implementations, the hardware components of machine learning infrastructure 600 can provide computational power, storage capabilities, memory capacity and networking support necessary for training, inference, and deployment of complex models efficiently. The choice of hardware infrastructure components 610 can vary depending on factors such as the scale of the workload, performance requirements, budget constraints, and available expertise. The hardware components of machine learning infrastructure used for sequence modeling may include processing units 620. Processing units can include Central Processing Units (CPUs). CPUs are the primary processing units in a machine learning infrastructure. They handle tasks such as data preprocessing, model training, and inference. Modern CPUs with multiple cores and threads can be essential for parallelizing computations and speeding up training and inference tasks. For sequence modeling, CPUs can be used for tasks such as data preprocessing, feature extraction, and serving inference requests.

The hardware components of machine learning infrastructure used for sequence modeling may include Graphics Processing Units (GPUs). GPUs are specialized hardware accelerators designed to handle parallel computations, making them particularly well-suited for training deep learning models, including sequence models. GPUs excel at performing matrix operations, which are prevalent in the computations involved in neural network training.

Many deep learning frameworks, such as TensorFlow and PyTorch, have GPU-accelerated implementations that leverage the parallel processing capabilities of GPUs to speed up model training. The hardware components of machine learning infrastructure used for sequence modeling can include Tensor Processing Units (TPUs). TPUs are custom-built hardware accelerators developed by Google specifically for machine learning workloads. TPUs can offer even higher computational performance and energy efficiency compared to GPUs, especially for tasks involving large-scale parallel processing. TPUs are well-suited for training and inference tasks in sequence modeling. In addition to GPUs and TPUs, specialized hardware accelerators, such as application-specific integrated circuits (ASICs) or neuromorphic processors, can be used to optimize specific machine learning tasks or algorithms.

These accelerators offer improved performance, energy efficiency, and scalability for specialized machine learning applications. The hardware components of machine learning infrastructure used for sequence modeling can include Dedicated AI Accelerators. Some hardware vendors offer dedicated AI accelerators designed specifically for machine learning workloads. These accelerators, such as NVIDIA's AI-focused GPUs or Google's Edge TPU, are optimized for deep learning tasks and may offer performance advantages over general-purpose CPUs or GPUs. In sequence modeling infrastructure, dedicated AI accelerators can be used for both training and inference tasks, providing high performance and energy efficiency.

The hardware components of machine learning infrastructure used for sequence modeling can include Field-Programmable Gate Arrays (FPGAs). FPGAs are programmable hardware devices that can be customized to accelerate specific computations or algorithms. In machine learning infrastructure, FPGAs can be used to accelerate certain parts of the training or inference pipeline, such as data preprocessing, feature extraction, or custom operations. While less common than CPUs, GPUs, or TPUs in machine learning infrastructure, FPGAs can offer flexibility and potential performance benefits for specific use cases.

The hardware components of machine learning infrastructure used for sequence modeling can include Storage 630 and Memory 640. High-speed memory (RAM) and storage (SSDs or NVMe drives) are essential for storing and accessing large datasets, model parameters, and intermediate results during training and inference. Fast memory and storage subsystems help reduce data transfer bottlenecks and improve overall system performance, especially for large-scale sequence modeling tasks with massive datasets.

The hardware components of machine learning infrastructure used for sequence modeling can also include Networking Infrastructure 650. High-speed networking infrastructure is crucial for distributed training and serving of machine learning models. Fast and reliable interconnects between compute nodes enable efficient data sharing and communication during distributed training tasks, providing the capacity to scale sequence modeling workloads across multiple machines or clusters. Cloud-based machine learning infrastructure can be also an alternative choice built on a combination of compute, storage, networking, and specialized machine learning hardware.

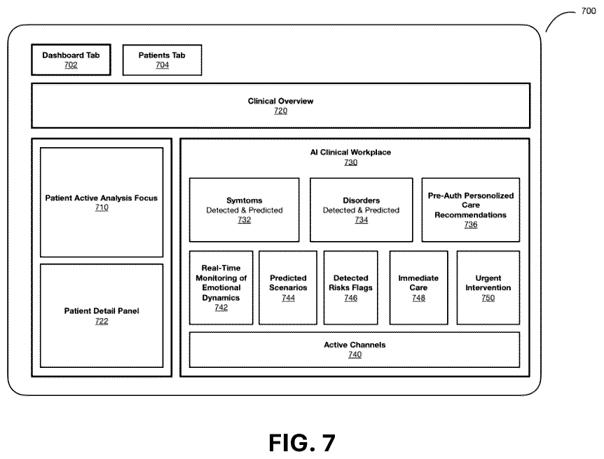

FIG. 7 illustrates a screenshot of a sample real-time output interface and user-interaction system or user interface 700 for a mental health care application according to some implementations. The interface may include a dashboard tab serving as a command center for clinicians, providing instant situational awareness via a multi-zone layout optimized for tablet workflows. The interface may include a clinical overview section with multiple clickable clinical metrics cards that enable one-click navigation to filtered patient lists and detailed analytics. The interface includes components for the AI Clinical Workspace, featuring eight selectable cards arranged in two rows. The top row includes three primary components: Symptoms, Disorders, and Pre-Auth Personalized Care Recommendations. The bottom row includes five micro-components:
Real-Time Monitoring of Emotional Dynamics, Predicted Scenarios, Detected Risks Flags, Immediate Care, and Urgent Intervention. Each card is configured to open a corresponding detailed analysis modal when selected. The interface may include a display of Active Channels, where each channel represents a multi-modal data integration system synthesizing information from input sequences. Additionally, the interface may include two stacked components: the Patient Active Analysis Focus and the Patient Detail Panel, the latter presenting real-time vital signs and patient-specific analytics.

The interface is structured to include multiple interactive components, providing a comprehensive clinical overview 720. This interface serves as an operational platform facilitating the integration of data analysis, user interaction, and clinical decision-making processes. The user interface 700 features several primary tabs, including a dashboard tab 702 and a patient's tab 704. These tabs may provide navigation options, enabling users to switch between different views or datasets within the application. The clinical overview 720 occupies a prominent position, potentially summarizing key data insights and alerts relevant to a patient's emotional and mental health status. Within the patient data section, the interface includes distinct elements such as a patient active analysis focus 710 and a patient detail panel 722. The patient active analysis focus 710 may provide real-time tracking of current analytical priorities, while the patient detail panel 722 offers comprehensive information about the individual under analysis, potentially including personal metrics and history. The AI clinical workplace 730 is embedded within the interface and comprises several critical functional components. These include elements for detecting and predicting symptoms 732 and disorders 734. These components are configured to analyze collected patient's dataset and provide insights related to mental-health contexts. Pre-auth personalized care recommendations 736 offer tailored, personalized, evidence-informed, and patient-specific treatment suggestions based on the system's detection and predictive analysis. Further, the interface incorporates active channels 740, comprising functionalities like real-time monitoring of emotional dynamics 742, predicted scenarios 744, detected risks flags 746, immediate care 748, and urgent intervention 750. These components enable dynamic oversight and swift response to early detection and prediction analysis, ensuring timely intervention and care adjustments as necessitated by the detected, and predicted indicators. Overall, the user interface 700 enables a user-centric interaction with an emphasis on proactive intervention and comprehensive data visualization in mental health management.

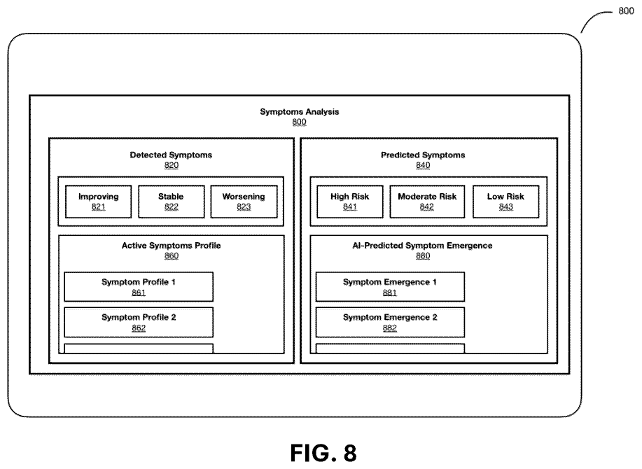

FIG. 8 illustrates a screenshot of a sample real-time output interface and user-interaction system showing the Symptoms Analysis window according to some implementations. The window features a dual-tab interface separating Detected Symptoms from Predicted Symptoms. The Detected Symptoms tab displays Active Symptoms Profile, sorted by severity (e.g., Moderate to Mild), with trend indicators (e.g., worsening, stable, improving) to provide evidence-based tracking. The Predicted Symptoms tab presents AI-Predicted Symptom Emergence as AI-generated predictions of potential future symptoms, sorted by risk level (e.g., High to Low), facilitating comprehension of complex machine-learning outputs for clinical decision-making.

In some implementations, a symptoms analysis framework 800 may comprise various components to analyze detected and predicted symptoms. The framework is divided into two main sections: Detected Symptoms 820 and Predicted Symptoms 840. The Detected Symptoms 820 section includes classifications of detected symptoms as improving 821, stable 822, or worsening 823. This categorization enables the system to track changes in symptoms over time and assess the current state of emotional health. The Predicted Symptoms 840 section categorizes the predicted symptoms based on risk levels, namely high risk 841, moderate risk 842, and low risk 843. This classification may assist in identifying potential future challenges by predicting the likelihood of specific emotional conditions emerging or escalating. Beneath these sections, the framework includes an Active Symptoms Profile 860, which may offer a detailed listing of present symptoms through entries such as Symptom Profile 1 861 and Symptom Profile 2 862. These profiles can be structured to provide real-time insights into ongoing symptoms states. The framework also contains an AI-Predicted Symptom Emergence 880 section, which identifies anticipated symptoms such as Symptom Emergence 1 881 and Symptom Emergence 2 882. These categories allow for the forecasting of potential new symptoms, potentially enabling preemptive interventions. Overall, the symptoms analysis framework 800 facilitates comprehensive monitoring by integrating current status and predictive capabilities, supporting both real-time assessments and forward-looking strategies in mental health care.

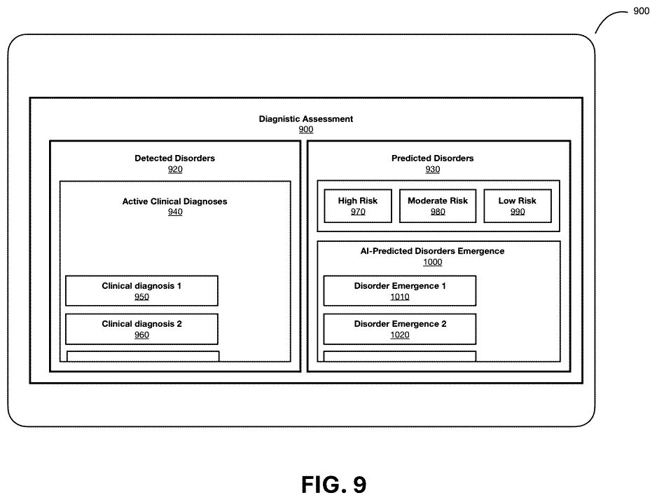

FIG. 9 illustrates a screenshot of a sample real-time output interface and user-interaction system showing the Diagnostic Assessment window according to some implementations. In some implementations, a diagnostic assessment interface 900 may be configured to organize elements pertinent to mental health evaluation.

The interface is divided into two primary categories: detected disorders 920 and predicted disorders 930. The detected disorders 920 section further categorizes data into active clinical diagnoses 940, which can include multiple clinical diagnoses such as clinical diagnosis 1(950) and clinical diagnosis 2(960). These elements indicate currently identified conditions within the individual's diagnostic profile.

The window features a dual-tab interface separating Detected Disorders from Predicted Disorders. The Detected Disorders tab displays multiple active clinical diagnoses with comprehensive diagnostic details and full diagnostic accountability. The Predicted Disorders tab presents AI-Predicted Disorders Emergence as AI-predicted potential future diagnoses, sorted by risk priority, enabling proactive clinical assessment and decision-making.

The predicted disorders 930 section is structured to assess risk levels associated with potential future disorders. It classifies risks into three distinct levels: high risk 970, moderate risk 980, and low risk 990. This allows for stratified prediction of disorder emergence, catering to varied intervention strategies.

Additionally, the predicted disorders 930 section integrates AI-predicted disorders emergence 1000. This component provides anticipatory data on potential disorders developments. It can include one or more disorders such as disorder emergence 1 (1010) and disorder emergence 2 (1020), offering insights into disorder patterns that may manifest based on current data analytics.

The diagnostic assessment interface 900 may facilitate comprehensive evaluation by enabling visualization of current detected diagnoses alongside predictive analytics. The structure supports the system's goals of enhancing proactive intervention and personalized care plans by continuously monitoring and analyzing emotional dynamics. This framework allows for adaptive mental health management by recognizing and anticipating potential mental health challenges.

Computer-based methods for analysis trained using machine learning, may comprise: collecting, by a first computing device, a plurality of contemporaneous Instant Emotional State Information (IESI) over time, from an individual, through one or multiple pre-existing audio-video Emotion Recognition System (ERS); capturing, by a second computing device, contemporaneous further data, from the same individual; training, by a sequence modeling system, weights using plurality of contemporaneous IESI, and further data, wherein the weights are learned from both contemporaneous IESI and further data through multi-task learning, and wherein the learning facilitates early detection, prediction, and proactive intervention in mental health care. The method may comprise collecting, by the first computing device, further contemporaneous IESI based on audio-only and video-only through different channels. The computing device learns weights using the further IESI, wherein the weights are learned through multi-task learning. The computing device analyzes the further IESI using the learned weights to perform further analysis including consistency examination, as well as to facilitate early detection, prediction, and proactive intervention in mental health care, based on the further IESI. In some implementations, the system may work in absence of further IESI.

Further data can include various contemporaneous physiological data. The further data can include one or more of contemporaneous physiological data.

The method may comprise early detection of emotional patterns, indicators, trends and risks in mental health care. The method may comprise early detection of emotional patterns, indicators, trends and risks, prediction, and proactive intervention in mental health care using analysis and predictions of emotional patterns, indicators, trends, and risks.

A computing device may collect a plurality of Instant Emotional State Information (IESI) over time from one or multiple pre-existing audio-video Emotion Recognition System (ERS), wherein the pre-existing audio-video ERS can be additionally benefited from further modalities including various contemporaneous physiological data.

The computing device and the second computing device may be the same computing device.

In some implementations, the systems and methods herein may be performed by a computer programing product comprising code which causes one or more processes to perform their operations.

While the invention has been described in detail with reference to preferred embodiments, various modifications and enhancements thereon will become apparent to those skilled in the art. Therefore, the examples provided should not limit the spirit and scope of the present invention; but rather should be understood in the broadest sense allowable by law.

1. A method for analyzing, identifying, and predicting emotional features related to proactive intervention, the method comprising:
collecting, into a sequence-modeling computing system, a plurality of information channels comprising contemporaneous Instant Emotional State Information (IESI) captured over time from an individual and further data from the individual;
training, by the sequence-modeling computing system, a set of model parameters using the plurality of information channels, wherein the set of model parameters are jointly learned through a multi-task learning process applied across the plurality of information channels; and
performing machine-learning analysis configured to generate at least one detection, prediction, or intervention-related output and to facilitate at least one early detection, prediction, and proactive intervention in mental-health contexts based on the IESI and the further data.

2. The method of claim 1, wherein the training facilitates early detection.

3. The method of claim 1, wherein the training facilitates prediction.

4. The method of claim 1, wherein the IESI includes an emotional type or an intensity of the emotional type.

5. The method of claim 1, wherein the further data includes contemporaneous physiological data.

6. The method of claim 1, wherein the plurality of information channels further comprises further IESI.

7. A system for analyzing emotional dynamics, comprising:
a sequence-modeling computing system configured
to receive a plurality of information channels including Instant Emotional State Information (IESI) from an individual and further data from one or more sensors associated with the individual,
to perform a multi-task learning process including a set of model parameters using the plurality of information channels, wherein the set of model parameters are jointly trained across the plurality of information channels; and
to perform machine-learning analysis configured to generate at least one detection, prediction, or intervention-related output and to facilitate at least one early detection, prediction, and proactive intervention in mental-health contexts based on the IESI and the further data.

8. The system of claim 7, wherein detection includes detection of one or more emotional features.

9. The system of claim 7, wherein prediction includes prediction of one or more emotional features.

10. The system of claim 7, wherein intervention includes initiating corrective or supportive recommendation before clinical deterioration occurs.

11. A method for detecting and predicting emotional features for at least one early detection, prediction, and proactive intervention in mental-health contexts, the method comprising:
real-time monitoring of one or more emotional features;
deriving one or more real-time insights based on the one or more emotional features;
generating one or more predictive analytics from one or more emotional features and derived one or more insights;
identifying one or more target emotional indicators, trends, patterns, and risks based on the one or more emotional features and derived one or more insights;
predicting one or more target emotional indicators, trends, patterns, and risks based on the one or more predictive analytics;
generating at least one alert or flag signal based on identified or predicted one or more target emotional indicators, trends, patterns, and risks;
generating one or more proactive intervention recommendations;
generating one or more care-related guidance or recommendations; generating one or more personalized or adaptive care plans;
constructing one or more real-time analytical summaries based on one or more real-time insights the generated one or more proactive intervention recommendations, one or more care-related guidance or recommendations, or one or more personalized or adaptive care plans;
displaying a visualization of analysis results of the one or more real-time analytical summaries; and
exporting or transmitting the analysis results in one or more data formats.

12. The method of claim 11, wherein the one or more emotional features include at least one of a pattern, an indicator, a trend, or a risk.

13. The method of claim 12, wherein a pattern includes at least one of a recurring or statistically significant combination of emotional, behavioral, linguistic, physiological, or cognitive signals detected over time.

14. The method of claim 12, wherein a pattern is at least one of a symptom-cluster pattern, disorder-like pattern, or recurrent behavioral regularities.

15. The method of claim 12, wherein an indicator is at least one of a discrete signal, cue, or measurable attribute that reflects an emotional or mental-state characteristic at a given time.

16. The method of claim 12, wherein an indicator is at least one of an emotional cue, physiological arousal, consistency or discrepancy markers, mismatch between verbal reports and expressed affect, or trigger-related emotional indications.

17. The method of claim 12, wherein a trend includes a directional or progressive change in emotional or behavioral features across multiple timepoints.

18. The method of claim 12, wherein a trend includes at least one of a symptom drift, a gradual movement from mild to moderate anxiety-related feature, an incremental accumulation of risk markers, a progressive increase in hopelessness indicators, improvement trajectories, or a reduction in avoidance-related signals.

19. The method of claim 12, wherein a risk includes at least one of a predicted probability or likelihood of an undesirable future mental-health-related outcome based on detected features, patterns, or trends.

20. The method of claim 12, wherein a risk includes at least one of acute risk, emergent indicators associated with crisis, self-harm propensity, relapse risk, feature constellations predictive of recurrence of a depressive or anxiety-related state, treatment-discontinuation risk, signals correlated with decreased engagement, escalation risk, or projections indicating progression toward severe impairment or functional decline.
